SEQUENCE LISTING (34 sequences)

SEQ ID NO: 1 - protein, 10 aa, Artificial Sequence
  Description of Artificial Sequence Synthetic peptide
GGGGSGGGGS

SEQ ID NO: 2 - protein, 5 aa, Artificial Sequence
  Description of Artificial Sequence Synthetic peptide
GGGGS

SEQ ID NO: 3 - protein, 5 aa, Artificial Sequence
  Description of Artificial Sequence Synthetic peptide
EAAAK

SEQ ID NO: 4 - protein, 46 aa, Artificial Sequence
  Description of Artificial Sequence Synthetic polypeptide
AEAAAKEAAAKEAAAKEAAAKALEAEAAAKEAAAKEAAAKEAAAKA

SEQ ID NO: 5 - protein, 5 aa, Artificial Sequence
  Description of Artificial Sequence Synthetic peptide
PAPAP

SEQ ID NO: 6 - protein, 12 aa, Artificial Sequence
  Description of Artificial Sequence Synthetic peptide
AEAAAKEAAAKA

SEQ ID NO: 7 - protein, 17 aa, Artificial Sequence
  Description of Artificial Sequence Synthetic peptide
VSQTSKLTRAETVFPDV

SEQ ID NO: 8 - protein, 6 aa, Artificial Sequence
  Description of Artificial Sequence Synthetic peptide
PLGLWA

SEQ ID NO: 9 - protein, 5 aa, Artificial Sequence
  Description of Artificial Sequence Synthetic peptide
RVLAE

SEQ ID NO: 10 - protein, 10 aa, Artificial Sequence
  Description of Artificial Sequence Synthetic peptide
EDVVCCSMSY

SEQ ID NO: 11 - protein, 8 aa, Artificial Sequence
  Description of Artificial Sequence Synthetic peptide
GGIEGRGS

SEQ ID NO: 12 - protein, 10 aa, Artificial Sequence
  Description of Artificial Sequence Synthetic peptide
TRHRQPRGWE

SEQ ID NO: 13 - protein, 10 aa, Artificial Sequence
  Description of Artificial Sequence Synthetic peptide
AGNRVRRSVG

SEQ ID NO: 14 - protein, 9 aa, Artificial Sequence
  Description of Artificial Sequence Synthetic peptide
RRRRRRRRR

SEQ ID NO: 15 - protein, 4 aa, Artificial Sequence
  Description of Artificial Sequence Synthetic peptide
GFLG

SEQ ID NO: 16 - protein, 11 aa, Artificial Sequence
  Description of Artificial Sequence Synthetic peptide
GGSGGHMGSGG

SEQ ID NO: 17 - protein, 8 aa, Artificial Sequence
  Description of Artificial Sequence Synthetic peptide
GGSGGGGG

SEQ ID NO: 18 - protein, 11 aa, Artificial Sequence
  Description of Artificial Sequence Synthetic peptide
GGSGGSGGSGG

SEQ ID NO: 19 - protein, 8 aa, Artificial Sequence
  Description of Artificial Sequence Synthetic peptide
SGGGSSHS

SEQ ID NO: 20 - protein, 10 aa, Artificial Sequence
  Description of Artificial Sequence Synthetic peptide
SGGSGGSSHS

SEQ ID NO: 21 - protein, 13 aa, Artificial Sequence
  Description of Artificial Sequence Synthetic peptide
SGGSGGSGGSSHS

SEQ ID NO: 22 - protein, 5 aa, Artificial Sequence
  Description of Artificial Sequence Synthetic peptide
GGSGG

SEQ ID NO: 23 - protein, 16 aa, Artificial Sequence
  Description of Artificial Sequence Synthetic peptide
GGGGSLVPRGSGGGGS

SEQ ID NO: 24 - protein, 18 aa, Artificial Sequence
  Description of Artificial Sequence Synthetic peptide
GGGSEGGGSEGGGSEGGG

SEQ ID NO: 25 - protein, 8 aa, Artificial Sequence
  Description of Artificial Sequence Synthetic peptide
AAGAATAA

SEQ ID NO: 26 - protein, 5 aa, Artificial Sequence
  Description of Artificial Sequence Synthetic peptide
GGGGG

SEQ ID NO: 27 - protein, 5 aa, Artificial Sequence
  Description of Artificial Sequence Synthetic peptide
GGSSG

SEQ ID NO: 28 - protein, 11 aa, Artificial Sequence
  Description of Artificial Sequence Synthetic peptide
GSGGGTGGGSG

SEQ ID NO: 29 - protein, 15 aa, Artificial Sequence
  Description of Artificial Sequence Synthetic peptide
GGGGSGGGGSGGGGS

SEQ ID NO: 30 - protein, 15 aa, Artificial Sequence
  Description of Artificial Sequence Synthetic peptide
EAAAKEAAAKEAAAK

SEQ ID NO: 31 - protein, 59 aa, Artificial Sequence
  Description of Artificial Sequence Synthetic polypeptide
GDRVADVIESSIGDSVSRALTHALPAPTGQNTQVSSHRLDTGKVPALQAAEIGASSNAS

SEQ ID NO: 32 - DNA, 21 bp, Artificial Sequence
  Description of Artificial Sequence Synthetic primer
gactggttccaattgacaagc

SEQ ID NO: 33 - DNA, 21 bp, Artificial Sequence
  Description of Artificial Sequence Synthetic primer
gcaaatggcattctgacatcc

SEQ ID NO: 34 - protein, 6 aa, Artificial Sequence
  Description of Artificial Sequence Synthetic 6xHis tag
HHHHHH

(19) United States
(12) Patent Application Publication
(10) Pub. No.: US 2016/0185826 A1
(43) Pub. Date: Jun. 30, 2016

(54) VIRUS-LIKE PARTICLE VACCINES

(71) Applicant: Medigen Biotechnology Corp., Taipei City (TW)

(72) Inventors: Young-Sun LIN, Taipei City (TW); Jinyi CHENG, Taipei City (TW); Ya-Lin CHIANG, New Taipei City (TW); Ming-Cheng CHEN, New Taipei City (TW); Kuei-Tai A. LAI, New Taipei City (TW); Chih Ya YANG, Taipei City (TW)

(73) Assignee: Medigen Biotechnology Corp., Taipei City (TW)

(21) Appl. No.: 15/015,249

(22) Filed: Feb. 4, 2016

Related U.S. Application Data
(63) Continuation-in-part of application No. 14/819,684, filed on Aug. 6, 2015.
(60) Provisional application No. 62/034,475, filed on Aug. 7, 2014.

(51) Int. Cl. C07K 14/005; A61K 39/12; A61K 39/29; A61K 39/125; A61K 39/15; C12N 7/00; A61K 39/145

(52) U.S. Cl. CPC C07K 14/005; C12N 7/00; A61K 39/12; A61K 39/145; A61K 39/125; A61K 39/15; A61K 39/292; A61K 39/29; C12N 2770/16071; C12N 2770/16034; C12N 2770/16023; C12N 2770/16022; C12N 2760/16171; C12N 2760/16134; C12N 2760/16123; C12N 2760/16122; C12N 2770/32371; C12N 2770/32334; C12N 2770/32323; C12N 2770/32322; C12N 2720/12371; C12N 2720/12334; C12N 2720/12323; C12N 2720/12322; C07K 2319/40; A61K 2039/5258

(57) ABSTRACT

The invention is directed to dimeric fusion proteins and virus-like particles comprising such dimeric fusion proteins. These dimeric fusion proteins comprise an antigen or antigenic fragment carried between two viral structural proteins or fragments thereof, with or without linkers, in a manner that, relative to traditional monomeric platforms, minimizes steric hindrance among the antigen or antigenic fragment and the viral structural proteins or fragments thereof. This novel design provides for multivalent vaccines and enhanced immunogenicity. The invention also relates to nucleic acids encoding such dimeric fusion proteins and host cells comprising such nucleic acids. The invention further relates to pharmaceutical compositions comprising the dimeric fusion proteins and/or virus-like particles of the invention, and methods of prevention or treatment using such compositions.

### RELATED APPLICATIONS

This application is a continuation-in-part of U.S. application Ser. No. 14/819,684, filed Aug. 6, 2015, which claims the benefit of U.S. Provisional Application No. 62/034,475, filed Aug. 7, 2014, all of which are incorporated herein by reference.

### SEQUENCE LISTING

The instant application contains a Sequence Listing which has been submitted electronically in ASCII format and is hereby incorporated by reference in its entirety. Said ASCII copy, created on Sep. 21, 2015, is named 12677.0001_SL.txt and is 8,900 bytes in size.

### FIELD OF THE INVENTION

The present invention relates to the fields of virology, immunology, microbiology, molecular biology, biochemistry, and genetics. In particular, the present invention relates to immunogenic compositions comprising virus-like particles comprising fusion proteins comprising antigenic peptide sequences of pathogens, viral structural peptides which may or may not themselves be immunogenic, and, optionally, one or more linkers associated with the antigen or antigenic fragment and the viral structural proteins. Methods of eliciting an immune response with the fusion proteins of the invention are also described.

### BACKGROUND OF THE INVENTION

Vaccines typically comprise attenuated viruses, or other attenuated microorganisms, or combinations thereof. Though such vaccines may produce strong immune responses, they bear the risk of reverting to infectious forms that may harm the patient. Existing vaccines that comprise recombinant antigens carry less risk of infection, but they often provide weaker immune responses. One reason these weaker immune responses occur is because traditional recombinant vaccines hinder the ability of recombinant antigens to adopt a conformation that would generate an optimal immune response in the subject receiving the vaccine.

Virus-like particles (VLPs) are morphologically and structurally similar to viruses, providing a platform for presenting proteins on the VLP surface in a highly immunogenic form. Although VLPs comprise viral structural proteins, they do not comprise viral genomic material.

Compared to other vaccine platforms, VLPs have several advantages. First, VLPs are safer than live or attenuated vaccines, as VLPs lack infectious genetic material, can be designed to exclude immunosuppressive viral proteins, and cannot revert to an infectious state. Second, VLPs are readily produced in non-mammalian cell lines, thus increasing production speed, scalability, and cost-effectiveness. Third, VLPs are typically able to induce consistently high levels of neutralizing antibodies, even without adjuvants, at least in part due to their highly ordered structure, which facilitates the presentation of epitopes. Fourth, VLPs can serve as display platforms for heterologous antigens. Fifth, VLPs often can break B cell tolerance and induce self-regulated auto-antibodies. Sixth, VLPs carry antigenic epitopes to both the MHC class I and class II pathways.

Because they confer such advantages, virus-like particles have been used as a platform for attachment or display of foreign antigens to the immune system. However, in the traditional approach, an antigen is fused to a single viral structural protein, often inside a loop structure of the viral structural protein. Such configurations are referred to herein as “monomeric fusion proteins.” The term “monomeric fusion protein” refers to an antigen or antigenic fragment fused to the N- or C-terminus of a single viral structural protein, or an antigen or antigenic fragment fused within a loop region of a single viral structural protein. Such monomeric configurations interfere with antigen folding, particularly in the case of larger antigens or antigens comprising more complex folding patterns, and have led to little success because they do not adequately maintain the native antigen conformation. Antigen conformation comprising or resembling native conformation plays an important role in immune system recognition and many antigens and antigenic fragments cannot maintain or sufficiently resemble their native conformation when present in a monomeric fusion protein. Therefore, there is a need to develop a new virus-like particle vaccine platform.

### SUMMARY OF THE INVENTION

The present invention differs from the traditional virus-like particle platforms because it does not utilize a monomeric fusion protein design. Instead, the present invention comprises a non-monomeric fusion protein design, such as, for example, a dimeric fusion protein design. The term “dimeric fusion protein” refers to an antigen or antigenic fragment in which the N-terminus of the antigen is fused, with or without a linker, to the C-terminus of an N-terminal viral structural protein (i.e., a viral structural protein that is positioned N-terminally with respect to the antigen), and the C-terminus of the antigen is fused, with or without a linker, to the N-terminus of a C-terminal viral structural protein (i.e., a viral structural protein that is positioned C-terminally with respect to the antigen). The fusion proteins of the present invention comprise antigens or antigenic fragments in conformations that enhance or otherwise optimize a subject's immune response compared to prior art platforms. In certain embodiments, the conformations structurally resemble the conformation that the antigen or antigenic fragment exhibits under natural or other nonrecombinant circumstances. The antigen-presenting platform of the present invention facilitates folding of antigens, or fragments thereof, into conformations that comprise or resemble their native conformation, by reducing or otherwise affecting steric and other influences that either oppose or are less than optimal for such folding.

The present invention also differs from traditional virus-like particle platforms because it does not utilize a design in which the antigen or antigenic fragment is either inserted into a loop region of a viral structural protein or fused to a single viral structural protein terminus. Instead, the present invention comprises an antigen carried between two viral structural proteins or fragments thereof, with or without linkers, such that the viral structural proteins and optional linkers will not be affected by the presence of the antigen or hinder antigen folding into a conformation comprising or resembling native conformation. In some embodiments, the viral structural proteins and/or optional linkers will facilitate antigen folding into a conformation comprising or resembling native conformation.

The design of the present invention facilitates antigen folding into a conformation comprising or resembling native conformation, which helps induce an immune response in a subject. This occurs at least in part because antigens and antigenic fragments of the present invention are more likely to be displayed in a conformation comprising or resembling native conformation in the context of the fusion proteins and resulting VLPs of the present invention.

The design of the present invention facilitates the assembly of antigenic fusion proteins into a VLP structure with improved immunogenicity over traditional platforms. This occurs at least in part because the antigen or antigenic fragment in the present invention is less likely to hinder folding of the viral structural proteins, or vice versa, or a combination thereof, than when the antigen exists in a loop region of the viral structural protein or when the antigen or antigenic fragment is attached to the N- or C-terminus of the viral structural protein, as in prior art designs.

The enhanced ability of the fusion proteins of the present invention to fold into conformations comprising or resembling native conformations also enables the present invention to produce multivalent vaccines and enhanced immunogenicity against the viral structural proteins in addition to the antigen.

In the present invention, optimized immunogenicity and improved potential for multivalence are unexpectedly found in a novel platform comprising recombinant fusion proteins comprising an antigen, wherein the N-terminus of the antigen is fused, with or without a linker, to an N-terminal viral structural protein, and the C-terminus of the antigen is fused, with or without a linker, to a C-terminal viral structural protein, and in which the viral structural proteins are, independently or together, capable of forming a virus-like particle. Fusion proteins of the present invention are capable of forming novel VLP platforms capable of displaying the antigen in a conformation comprising or resembling its native conformation and in a stable and repetitive manner, thus working as an effective vaccine producing T- and/or B-cell-mediated immunity.

In one embodiment, the present invention provides a fusion protein comprising V1-L1-Ag-L2-V2, wherein V1 is an N-terminal viral structural protein, L1 is an N-terminal linker, Ag is an antigen or antigenic fragment of a pathogen, L2 is a C-terminal linker, and V2 is a C-terminal viral structural protein, and wherein each of V1 and V2 is, independently or together, capable of forming a virus-like particle.

In one embodiment, the present invention provides a fusion protein comprising V1-L1-Ag-V2 or V1-Ag-L2-V2, wherein V1 is an N-terminal viral structural protein, L1 is an N-terminal linker, Ag is an antigen or antigenic fragment of a pathogen, L2 is a C-terminal linker, and V2 is a C-terminal viral structural protein, and wherein each of V1 and V2 is, independently or together, capable of forming a virus-like particle.

In one embodiment, the present invention provides a fusion protein comprising V1-Ag-V2, wherein V1 is an N-terminal viral structural protein, Ag is an antigen or antigenic fragment of a pathogen, V2 is a C-terminal viral structural protein, and wherein each of V1 and V2 is, independently or together, capable of forming a virus-like particle.

In one embodiment, the present invention provides a fusion protein comprising V1-L1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein V1 and V2 are the same.

In one embodiment, the present invention provides a fusion protein comprising V1-L1-Ag-V2 or V1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein V1 and V2 are the same.

In one embodiment, the present invention provides a fusion protein comprising V1-Ag-V2, wherein V1 is a fragment of an N-terminal viral structural protein, Ag is an antigen or antigenic fragment of a pathogen, V2 is a fragment of a C-terminal viral structural protein, and wherein each of V1 and V2 is, independently or together, capable of forming a virus-like particle, and wherein V1 and V2 are the same.

In one embodiment, the present invention provides a fusion protein comprising V1-L1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein V1 and V2 are fragments of different proteins from the same virus.

In one embodiment, the present invention provides a fusion protein comprising V1-L1-Ag-V2 or V1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein V1 and V2 are fragments of different proteins from the same virus.

In one embodiment, the present invention provides a fusion protein comprising V1-Ag-V2, wherein V1 is a fragment of an N-terminal viral structural protein, Ag is an antigen or antigenic fragment of a pathogen, V2 is a fragment of a C-terminal viral structural protein, and wherein each of V1 and V2 is, independently or together, capable of forming a virus-like particle, and wherein V1 and V2 are fragments of different proteins from the same virus.

In one embodiment, the present invention provides a fusion protein comprising V1-L1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein V1 and V2 are fragments of proteins of different viruses.

In one embodiment, the present invention provides a fusion protein comprising V1-L1-Ag-V2 or V1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein V1 and V2 are fragments of proteins from different viruses.

In one embodiment, the present invention provides a fusion protein comprising V1-Ag-V2, wherein V1 is a fragment of an N-terminal viral structural protein, Ag is an antigen or antigenic fragment of a pathogen, V2 is a fragment of a C-terminal viral structural protein, and wherein each of V1 and V2 is, independently or together, capable of forming a virus-like particle, and wherein V1 and V2 are fragments of proteins from different viruses.

In one embodiment, the present invention provides a fusion protein comprising V1-L1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein the fragments are from different portions of the same parent viral structural protein, and wherein the combined amino acid sequence of V1 and V2 comprises less than the complete amino acid sequence of the parent viral structural protein.

In one embodiment, the present invention provides a fusion protein comprising V1-L1-Ag-V2 or V1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein the fragments are from different portions of the same parent viral structural protein, and wherein the combined amino acid sequence of V1 and V2 comprises less than the complete amino acid sequence of the parent viral structural protein.

In one embodiment, the present invention provides a fusion protein comprising V1-Ag-V2, wherein V1 is a fragment of an N-terminal viral structural protein, Ag is an antigen or antigenic fragment of a pathogen, V2 is a fragment of a C-terminal viral structural protein, and wherein each of V1 and V2 is, independently or together, capable of forming a virus-like particle, and wherein the fragments are from different portions of the same parent viral structural protein, and wherein the combined amino acid sequence of V1 and V2 comprises less than the complete amino acid sequence of the parent viral structural protein.

In one embodiment, the present invention provides a fusion protein comprising V1-L1-Ag-L2-V2, wherein V1 is an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein V2 is a fragment of V1.

In one embodiment, the present invention provides a fusion protein comprising V1-L1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a C-terminal viral structural protein, and wherein each of V1 and V2 is, independently or together, capable of forming a virus-like particle, and wherein V1 is a fragment of V2.

In one embodiment, the present invention provides a fusion protein comprising V1-L1-Ag-V2 or V1-Ag-L2-V2, wherein V1 is an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein V2 is a fragment of V1.

In one embodiment, the present invention provides a fusion protein comprising V1-L1-Ag-V2 or V1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a C-terminal viral structural protein, and wherein each of V1 and V2 is, independently or together, capable of forming a virus-like particle, and wherein V1 is a fragment of V2.

In one embodiment, the present invention provides a fusion protein comprising V1-Ag-V2, wherein V1 is an N-terminal viral structural protein, Ag is an antigen or antigenic fragment of a pathogen, V2 is a fragment of a C-terminal viral structural protein, and wherein each of V1 and V2 is, independently or together, capable of forming a virus-like particle, and wherein V2 is a fragment of V1.

In one embodiment, the present invention provides a fusion protein comprising V1-Ag-V2, wherein V1 is a fragment of an N-terminal viral structural protein, Ag is an antigen or antigenic fragment of a pathogen, V2 is a C-terminal viral structural protein, and wherein each of V1 and V2 is, independently or together, capable of forming a virus-like particle, and wherein V1 is a fragment of V2.

In one embodiment, the present invention provides a fusion protein comprising V1-L1-Ag-L2-V2, wherein V1 is an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein V2 is a fragment of a different protein from the same virus as V1.

In one embodiment, the present invention provides a fusion protein comprising V1-L1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a C-terminal viral structural protein, and wherein each of V1 and V2 is, independently or together, capable of forming a virus-like particle, and wherein V1 is a fragment of a different protein from the same virus as V2.

In one embodiment, the present invention provides a fusion protein comprising V1-L1-Ag-V2 or V1-Ag-L2-V2, wherein V1 is an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein V2 is a fragment of a different protein from the same virus as V1.

In one embodiment, the present invention provides a fusion protein comprising V1-L1-Ag-V2 or V1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a C-terminal viral structural protein, and wherein each of V1 and V2 is, independently or together, capable of forming a virus-like particle, and wherein V1 is a fragment of a different protein from the same virus as V2.

In one embodiment, the present invention provides a fusion protein comprising V1-Ag-V2, wherein V1 is an N-terminal viral structural protein, Ag is an antigen or antigenic fragment of a pathogen, V2 is a fragment of a C-terminal viral structural protein, and wherein each of V1 and V2 is, independently or together, capable of forming a virus-like particle, and wherein V2 is a fragment of a different protein from the same virus as V1.

In one embodiment, the present invention provides a fusion protein comprising V1-Ag-V2, wherein V1 is a fragment of an N-terminal viral structural protein, Ag is an antigen or antigenic fragment of a pathogen, V2 is a C-terminal viral structural protein, and wherein each of V1 and V2 is, independently or together, capable of forming a virus-like particle, and wherein V1 is a fragment of a different protein from the same virus as V2.

In one embodiment, the present invention provides a fusion protein comprising V1-L1-Ag-L2-V2, wherein V1 is an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein V2 is a fragment of a protein from a different virus than V1.

In one embodiment, the present invention provides a fusion protein comprising V1-L1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a C-terminal viral structural protein, and wherein each of V1 and V2 is, independently or together, capable of forming a virus-like particle, and wherein V1 is a fragment of a protein from a different virus than V2.

In one embodiment, the present invention provides a fusion protein comprising V1-L1-Ag-V2 or V1-Ag-L2-V2, wherein V1 is an N-terminal viral structural protein, L1 is an N-terminal linker, Ag is an antigen or antigenic fragment of a pathogen, L2 is a C-terminal linker, and V2 is a fragment of a C-terminal viral structural protein, and wherein each of V1 and V2 is, independently or together, capable of forming a virus-like particle, and wherein V2 is a fragment of a protein from a different virus than V1.

In one embodiment, the present invention provides a fusion protein comprising V1-L1-Ag-V2 or V1-Ag-L2-V2, wherein V1 is a fragment of an N-terminal viral structural protein, L1 is an N-terminal linker, Ag is an antigen or antigenic fragment of a pathogen, L2 is a C-terminal linker, and V2 is a C-terminal viral structural protein, and wherein each of V1 and V2 is, independently or together, capable of forming a virus-like particle, and wherein V1 is a fragment of a protein from a different virus than V2.

In one embodiment, the present invention provides a fusion protein comprising V1-Ag-V2, wherein V1 is an N-terminal viral structural protein, Ag is an antigen or antigenic fragment of a pathogen, V2 is a fragment of a C-terminal viral structural protein, and wherein each of V1 and V2 is, independently or together, capable of forming a virus-like particle, and wherein V2 is a fragment of a protein from a different virus than V1.

In one embodiment, the present invention provides a fusion protein comprising V1-Ag-V2, wherein V1 is a fragment of an N-terminal viral structural protein, Ag is an antigen or antigenic fragment of a pathogen, V2 is a C-terminal viral structural protein, and wherein each of V1 and V2 is, independently or together, capable of forming a virus-like particle, and wherein V1 is a fragment of a protein from a different virus than V2.

In one embodiment, the present invention provides any of the above fusion proteins, wherein Ag is selected from the group consisting of (a) an antigenic peptide, polypeptide, or protein from a viral pathogen, (b) an antigenic peptide, polypeptide, or protein from a bacterial pathogen, (c) an antigenic peptide, polypeptide, or protein from a parasitic pathogen, (d) an antigenic peptide, polypeptide, or protein from a fungal pathogen, and (e) an antigenic peptide, polypeptide, or protein from a prion.

In one embodiment, the present invention provides any of the above fusion proteins, wherein V1 and V2 are viral structural proteins from:
a. members of the family Adenoviridae (including, for example, members of the genera Atadenovirus, Aviadenovirus, lchtadenovirus, Mastadenovirus, and Siadenovirus);
b. members of the family Anelloviridae (including, for example, members of the genus Alphatorquevirus);
c. members of the family Arenaviridae (including, for example, members of the genus Arenavirus);
d. members of the family Arteriviridae (including, for example, members of the genus Arterivirus);
e. members of the family Astroviridae (including, for example, members of the genera Avian nephritis virus, Bovine astrovirus, Capreolus capreolus astrovirus, Chicken astrovirus, Duck astrovirus, Feline astrovirus, Human astrovirus, Mamastrovirus, Mink astrovirus, Ovine astrovirus, Porcine astrovirus, and Turkey astrovirus);
f. members of the family Bornaviridae (including, for example, members of the genus Bornavirus);
g. members of the family Bunyaviridae (including, for example, members of the genera Hantavirus, Nairovirus, Orthobunyavirus, Phlebovirus, and Tospovirus);
h. members of the family Caliciviridae (including, for example, members of the genera Lagovirus, Nebovirus, Norovirus, Sapovirus, and Vesivirus);
i. members of the family Coronaviridae (including, for example, members of the genera Alphacoronavirus, Betacoronavirus, Deltacoronavirus, and Gammacoronavirus);
j. members of the family Filoviridae (including, for example, members of the genera Cuevavirus, Ebolavirus, and Marburgvirus);
k. members of the family Flaviviridae (including, for example, members of the genera Hepacivirus, Flavivirus, Pegivirus, and Pestivirus);
l. members of the family Hepadnaviridae (including, for example, members of the genera Avihepadnavirus and Orthohepadnavirus);
m. members of the family Hepeviridae (including, for example, members of the genera Orthohepevirus and Piscihepevirus);
n. members of the family Herpesviridae (including, for example, members of the genera Cytomegalovirus, Iltovirus, Lymphocryptovirus, Macavirus, Mardivirus, Muromegalovirus, Percavirus, Proboscivirus, Rhadinovirus, Roseolovirus, Scutavirus, Simplexvirus, and Varicellovirus);
o. members of the family Orthomyxoviridae (including, for example, members of the genera Influenza virus A, Influenza virus B, Influenza virus C, Isavirus, Quaranjavirus, and Thogotovirus);
p. members of the family Papillomaviridae (including, for example, members of the genera Alphapapillomavirus, Betapapillomavirus, Gammapapillomavirus, Deltapapillomavirus, Epsilonpapillomavirus, Etapapillomavirus, lotapapillomavirus, Kappapapillomavirus, Lambdapapillomavirus, Mupapillomavirus, Nupapillomavirus, Omikronpapillomavirus, Pipapillomavirus, Thetapapillomavirus, Xipapillomavirus, and Zetapapillomavirus);
q. members of the family Paramyxoviridae (including, for example, members of the genera Aquaparamyxovirus, Avulavirus, Ferlavirus, Henipavirus, Metapneumovirus, Morbillivirus, Pneumovirus, Respirovirus, Rubulavirus, and TPMV-like viruses);
r. members of the family Parvoviridae (including, for example, members of the genera Ambidensovirus, Amdoparvovirus, Aveparvovirus, Bocaparvovirus, Brevidensovirus, Copiparvovirus, Dependoparvovirus, Erythroparvovirus, Hepandensovirus, Iteradensovirus, Penstyldensovirus, Protoparvovirus, Tetraparvovirus);
s. members of the family Picornaviridae (including, for example, members of the genera Aphthovirus, Aquamavirus, Avihepatovirus, Avisivirus, Cardiovirus, Cosavirus, Dicipivirus, Enterovirus, Erbovirus, Gallivirus, Hepatovirus, Hunnivirus, Kobuvirus, Kunsagivirus, Megrivirus, Mischivirus, Mosavirus, Oscivirus, Parechovirus, Pasivirus, Passerivirus, Rosavirus, Sakobuvirus, Salivirus, Sapelovirus, Senecavirus, Sicinivirus, Teschovirus, and Tremovirus);
t. members of the family Polyomaviridae (including, for example, members of the genera Polyomavirus, Avipolyomavirus, Orthopolyomavirus, and Wukipolyomavirus);
u. members of the family Poxviridae (including, for example, members of the genera Alphaentomopoxvirus, Avipoxvirus, Betaentomopoxvirus, Capripoxvirus, Cervidpoxvirus, Crocodylipoxvirus, Gammaentomopoxvirus, Leporipoxvirus, Molluscipoxvirus, Orthopoxvirus, Parapoxvirus, Suipoxvirus, and Yatapoxvirus);
v. members of the family Reoviridae (including, for example, members of the genera Aquareovirus, Cardoreovirus, Coltivirus, Cypovirus, Dinovernavirus, Fijivirus, Idnoreovirus, Mimoreovirus, Mycoreovirus, Orbivirus, Orthoreovirus, Oryzavirus, Phytoreovirus, Rotavirus, and Seadornavirus);
w. members of the family Rhabdoviridae (including, for example, members of the genera Cytorhabdovirus, Ephemerovirus, Lyssavirus, Novirhabdovirus, Nucleorhabdovirus, Perhabdovirus, Sigmavirus, Sprivivirus, Tibrovirus, Tupavirus, and Vesiculovirus); and
x. members of the family Togaviridae (including, for example, members of the genera Alphavirus and Rubivirus).

In one embodiment, the present invention provides any of the above fusion proteins, wherein V1 and V2 are viral VLP-forming polypeptides selected from, but not limited to: (a) HBc of HBV virus, (b) the small HBV-derived surface antigen (HBsAg), (c) the S domain of Norovirus capsid protein VP1, (d) the P domain of Norovirus capsid protein VP1, (e) Human Rotavirus VP2, (f) Human Rotavirus VP6, (g) the L1 major capsid protein of human papillomavirus; (h) the VP1 of human polyomavirus, (i) the VP1 of human JC virus, and (j) the VP2 of human adeno-associated virus 2, (k) the VP3 of human adeno-associated virus 2, (l) the S and P1 domain of Hepatitis E virus capsid protein VP1, and (m) the P2 domain of Hepatitis E virus capsid protein VP1.

In one embodiment, the present invention provides any of the above fusion proteins, wherein V1 and V2 are selected from the group consisting of: viral envelope proteins and viral capsid proteins. For example, Norovirus P, Norovirus S, and HBc are all capsid proteins, whereas HBs is a viral envelope protein. In one embodiment, V1 and V2 are both viral capsid proteins. In one embodiment, V1 and V2 are both envelope proteins. In one embodiment, one of V1 and V2 is a viral capsid protein, and the other of V1 and V2 is a viral envelope protein.

In one embodiment, the present invention provides any of the above fusion proteins, wherein, unless otherwise specified, V1 and V2 are the same viral structural protein.

In one embodiment, the present invention provides any of the above fusion proteins, wherein, unless otherwise specified, V1 and V2 are different viral structural proteins of the same virus.

In one embodiment, the present invention provides any of the above fusion proteins, wherein, unless otherwise specified, V1 and V2 are viral structural proteins of different viruses.

In one embodiment, the present invention provides any of the above fusion proteins, wherein at least one of V1 and V2 is immunogenic in the fusion protein, in the virus-like particle, or in both the fusion protein and the virus-like particle.

In one embodiment, the present invention provides any of the above fusion proteins, wherein both V1 and V2 are immunogenic in the fusion protein, in the virus-like particle, or in both the fusion protein and the virus-like particle.

In one embodiment, the present invention provides any of the above fusion proteins, wherein at least one of L1 and L2 is selected from the group consisting of: a flexible linker, a cleavable linker, a rigid linker, and an unstructured random coil peptide.

In one embodiment, the present invention provides any of the above fusion proteins, wherein L1 and L2 are the same linker.

In one embodiment, the present invention provides any of the above fusion proteins, wherein L1 and L2 are different linkers.

In one embodiment, the present invention provides a recombinant nucleic acid expression vector comprising a polynucleotide encoding any of the above fusion proteins.

In one embodiment, the present invention provides a host cell comprising a recombinant nucleic acid expression vector comprising a polynucleotide encoding any of the above fusion proteins.

In one embodiment, the present invention provides a virus-like particle comprising any of the above fusion proteins.

In one embodiment, the present invention provides a pharmaceutical composition comprising a virus-like particle comprising any of the above fusion proteins and a pharmaceutically acceptable carrier.

In one embodiment, the present invention provides a pharmaceutical composition comprising any of the above fusion proteins and a pharmaceutically acceptable carrier.

In one embodiment, the present invention provides a method of inducing an immune response in a mammalian subject comprising administering to the subject a pharmaceutical composition comprising a virus-like particle comprising any of the above fusion proteins and a pharmaceutically acceptable carrier in an amount sufficient to generate an immune response in the subject.

In one embodiment, the present invention provides a method of inducing an immune response in a mammalian subject comprising administering to the subject a pharmaceutical composition comprising any of the above fusion proteins and a pharmaceutically acceptable carrier in an amount sufficient to generate an immune response in the subject.

In one embodiment, the present invention provides a method for preparing virus-like particles, comprising culturing a host cell comprising a recombinant nucleic acid expression vector comprising a polynucleotide encoding any of the above fusion proteins under conditions that permit expression of said fusion protein and assembly of said fusion protein to form said virus-like particles.

### BRIEF DESCRIPTION OF THE DRAWINGS

The foregoing summary, as well as the following detailed description of the invention, will be better understood when read in conjunction with the appended drawings. For the purpose of illustrating the present invention, the attached drawings illustrate some, but not all, alternative embodiments. It should be understood, however, that the invention is not limited to the precise arrangements and instrumentalities shown. These figures, which are incorporated into and constitute part of the specification, assist in explaining the principles of the invention.

FIG. 1A illustrates a schematic structure of the virus-like-particle (VLP) template used in the Examples. As shown in FIG. 1A, the VLP template comprises one or more N- or C-terminal tags, two viral structural proteins (VP1 and VP2), an antigen, and linkers connecting the viral structural proteins to the antigen. FIG. 1A discloses “Hisx6” as SEQ ID NO: 34.

FIG. 1B depicts the linker systems used in the Examples.

FIG. 1B discloses SEQ ID NOs: 29, 29, 30, 30, and 31, respectively, in order of appearance.

FIGS. 2A-C illustrate the expression of the following recombinant VLP proteins: SP-GG (FIG. 2A), SP-GE (FIG. 2A), SP-GR (FIG. 2A), SP-EG (FIG. 2B), SP-EE (FIG. 2B), SP-ER (FIG. 2B), SP-RG (FIG. 2C), SP-RE (FIG. 2C), and SP-RR (FIG. 2C). The recombinant VLP proteins were expressed in Pichia pastoris GS115 by 1% methanol induction. After 24 h, the cell pellets were collected and homogenized by French press (20,000 psi, once). 10 μL of total (T), supernatant (S), or pellets (P) of homogenates were analyzed by Western blot using an anti-His tag primary antibody.

FIGS. 3A-D illustrate the expression of C-terminally-tagged VP1 (FIG. 3A) and the following recombinant C-terminally tagged VLP proteins: ΔSP-GG-VP1-C (FIG. 3B), ΔSP-GE-VP1-C (FIG. 3B), ΔSP-GR-VP1-C (FIG. 3B), ΔSP-EG-VP1-C (FIG. 3C), ΔSP-EE-VP1-C (FIG. 3C), ΔSP-ER-VP1-C (FIG. 3C), ΔSP-RG-VP1-C (FIG. 3D), ΔSP-RE-VP1-C (FIG. 3D), ΔSP-RR-VP1-C (FIG. 3D). The recombinant proteins were expressed in Pichia pastoris GS115 by 1% methanol induction. After 24 h, the cell pellets were collected and homogenized by French press (20,000 psi, once). 10 μL of total (T), supernatant (S), or pellets (P) of homogenates were analyzed by Western blot using an anti-EV71 VP1 primary antibody.

FIGS. 4A-B illustrate the expression of N-terminally-tagged HA1 (FIG. 4A) and the SP-EE-HA1 recombinant VLP protein (FIG. 4B). Expression of the following recombinant VLP proteins was also examined (data not shown): SP-GE-HA1, SP-GG-HA1, SP-GR-HA1, SP-EG-HA1, SP-ER-HA1, SP-RE-HA1, SP-RG-HA1, and SP-RR-HA1. The recombinant proteins were expressed in Pichia pastoris GS115 by 1% methanol induction. After 24 h, the cell pellets were collected and homogenized by French press (20,000 psi, once). 10 μL of total (T), supernatant (S), or pellets (P) of homogenates were analyzed by Western blot using an anti-His tag primary antibody.

FIG. 5 illustrates the expression of the SP-RE-M2e recombinant VLP protein. Expression of the following recombinant VLP proteins was also tested (data not shown): SP-GG-M2e, SP-GE-M2e, SP-GR-M2e, PS-EG-M2e, SP-EE-M2e, SP-ER-M2e, SP-RG-M2e, and SP-RR-M2e The recombinant VLP protein was expressed in Pichia pastoris GS115 by 1% methanol induction. After 24 h, the cell pellet was collected and homogenized by French press (20,000 psi, once). 10 μL of total (T), supernatant (S), or pellet (P) of the homogenate were analyzed by Western blot using an anti-His tag primary antibody. N=methanol-induced parental GS115.

FIG. 6 illustrates the expression of the SH-GR-VP1 recombinant VLP protein. Expression of the following recombinant VLP proteins was also tested (data not shown): SH-GG-VP1, SH-GE-VP1, SH-EG-VP1, SH-EE-VP1, SH-ER-VP1, SH-RG-VP1, SH-RE-VP1, and SH-RR-VP1. The recombinant VLP protein was expressed in Pichia pastoris GS115 by 1% methanol induction. After 24 h, the cell pellet was collected and homogenized by French press (20,000 psi, once). 10 μL of total (T), supernatant (S), or pellet (P) of the homogenate were analyzed by Western blot using an anti-VP1 primary antibody.

FIG. 7A illustrates the results from sucrose gradient analysis of VLPs produced from the following recombinant VLP proteins: SP-GG, SP-GE, SP-GR, SP-EG, SP-EE, SP-ER, SP-RG, SP-RE, SP-RR, ΔSP-GG-VP1-C, ΔSP-GE-VP1-C, ΔSP-GR-VP1-C, ΔSP-EG-VP1-C, ΔSP-EE-VP1-C, ΔSP-ER-VP1-C, ΔSP-RG-VP1-C, ΔSP-RE-VP1-C, ΔSP-RR-VP1-C.

FIG. 7B illustrates the results from sucrose gradient analysis of VLPs produced from the SP-EE-HA1 recombinant VLP protein.

FIG. 7C illustrates the results from sucrose gradient analysis of VLPs produced from the SP-RE-M2e recombinant VLP protein.

FIG. 7D illustrates the results from sucrose gradient analysis of VLPs produced from the SH-GR-VP1 recombinant VLP protein. Sucrose gradient analysis of VLPs produced form the following recombinant VLP proteins was also tested (data not shown): SP-GG-VP1, SP-GE-VP1, SP-GR-VP1, SP-EG-VP1, SP-EE-VP1, SP-ER-VP1, SP-RG-VP1, SP-RE-VP1, SP-RR-VP1, SP-GG-HA1, SP-GE-HA1, SP-GR-HA1, SP-EG-HA1, SP-ER-HA1, SP-RG-HA1, SP-RE-HA1, SP-RR-HA1, SP-GG-M2e, SP-GE-M2e, SP-GR-M2e, SP-EG-M2e, SP-EE-M2e, SP-ER-M2e, SP-RG-M2e, SP-RR-M2e, SH-GG-VP1, SH-GE-VP1, SH-EG-VP1, SH-EE-VP1, SH-ER-VP1, SH-RG-VP1, SH-RE-VP1, SH-RR-VP1, ΔSP-GG-VP1, ΔSP-GE-VP1, ΔSP-GR-VP1, ΔSP-EG-VP1, ΔSP-EE-VP1, ΔSP-ER-VP1, ΔSP-RG-VP1, ΔSP-RE-VP1, ΔSP-RR-VP1, ΔSP-GG-HA1-C, ΔSP-GE-HA1-C, ΔSP-GR-HA1-C, ΔSP-EG-HA1-C, ΔSP-EE-HA1-C, ΔSP-ER-HA1-C, ΔSP-RG-HA1-C, ΔSP-RE-HA1-C, and ΔSP-RR-HA1-C. The VLPs in supernatants of yeast homogenate were analyzed by 10-50% sucrose gradient (35,000 rpm for 4 h). Eleven fractions of 1 mL each were collected from top to bottom. The distribution of recombinant VLP proteins were analyzed by Western blot using anti-His tag or anti-VP1 primary antibodies, as indicated. IP=input. SP-(3×3), SP-(3×3)-HA1, SP-(3×3)-M2e, and SH-(3×3)-VP1 are constructs having an N-terminal tag. ΔSP-(3×3)-VP1-C is a construct having a C-terminal tag.

FIG. 8 illustrates an electron micrograph of SP-GG-VP1 virus-like particles (0.5 μg/μL Δ-SP-GG-VP1-C for 3 min, 1% UA for 30 sec). The protein samples were adsorbed on carbon-formvar-coated copper grids and negatively stained with 1% uranyl acetate aqueous solution. The grids were examined with a JEM-1230 electron microscope (JEOL Ltd., Tokyo, Japan) at 80 kV and 100 kV.

FIG. 9 illustrates a schematic structure of the virus-S-VP1-S like-particle (VLP) template used in the Examples. The VLP template comprises, from N- to C-terminus: N-terminal tags, a Norovirus S domain, a (G₄S)₂ flexible linker (SEQ ID NO: 1), EV71 VP1, a (G₄S)₂ flexible linker (SEQ ID NO: 1), a Norovirus S domain, a (G₄S)₂ flexible linker (SEQ ID NO: 1).

FIGS. 10A-B illustrate transmission electron micrographs of S-VP1-S virus-like particles. 8 μg of S-VP1-S VLPs were adsorbed onto a copper grid (300 mesh) for 3 min at room temperature. The grids were dried gently using filter paper. After staining with 1% uranyl acetate aqueous solution for 30 sec, the excess liquid was removed. The grids were examined with a JEM-1400 electron microscope at 80 kV.

FIG. 11 illustrates a Western blot analysis of antisera obtained from mice immunized with S-VP1-S VLPs. Using 20 μg of protein prepared from EV71-infected RD cell lysate as starting-material, Western blot analysis was performed using 1:500 and 1:1000 dilutions of antisera obtained from mice primed (1^{st}), boosted once (2^{nd}), and twice (3^{rd}) with 10 μg of S-VP1-S VLPs. A 1:1000 dilution of anti-VP1 monoclonal antibody (0.5 μg/μL, Abonova MAB1255-M05) was used as control.

FIG. 12 illustrates a graphical representation of the EV71 B4 neutralizing antibody titer in mice vaccinated with S-VP1-S VLPs. 50 μL of 100 TCID₅₀ of EV71 B4 was mixed with 50 μL of 2-fold serial diluted sera from mice immunized with VP1 only, S-S, and S-VP1-S proteins. Virus-sera mixtures were incubated at 37° C. for 2 hours and then added to 2×10⁴ cells/well of Vero cells. After 4 days of incubation, cells were fixed with 100 μL/well of 3.7% formaldehyde (diluted with 1×PBS). After 1 hour at room temperature (RT), cells were stained with crystal violate solution. Neutralization titer was determined by more than 50% of protection in such a dilution factor.

FIGS. 13A-F illustrate the expression of the following N-terminally tagged recombinant VLP proteins: SHBs-RG-VP1 (FIG. 13A); HBcHBs-GG-HA1 (FIG. 13B); HBsHBc-GG-HA1 (FIG. 13C); HBsP-GR-VP1 (FIG. 13D); HBsP-EE-HA1 (FIG. 13E); HBsHBs-GG-HA1 (FIG. 13F). The recombinant proteins were expressed in Pichia pastoris GS115 by 1% methanol induction. After 24 hours, the cell pellets were collected and homogenized by French press (20,000 psi, once). 10 μL of total (T), supernatant (S), or pellets (P) of homogenates were analyzed by Western blot using: an anti-EV71 VP1 primary antibody (FIGS. 13A, 13D); an anti-influenza H1N1 HA primary antibody (FIGS. 13B, 13C, 13F); an anti-H1N1 HA1 antiserum as primary antibody (FIG. 13E).

FIGS. 14A-B illustrate sucrose gradient analysis of HBcHBs-GG-VP1 virus-like particles. The expressed E. coli cell extract was loaded onto a 10-50% sucrose gradient ultracentrifuge tube (FIG. 14B). After 35,000 rpm ultracentrifugation (Beckman SW41 rotor) at 4° C. for 4 hours, 1 mL fractions were collected and analyzed by Western blot using anti-His tag antibody (FIG. 14A). “NC” indicates the negative control E. coli cell lysate. “PC” indicates the positive control HxSS-VP1 cell lysate.

FIG. 15 illustrates sucrose gradient analysis of HBsP-GR-VP1 virus-like particles. The VLPs in supernatants of yeast homogenate were analyzed by 10-50% sucrose gradient (35,000 rpm for 4 hours). Eleven fractions of 1 mL each were collected from top to bottom. The distribution of recombinant VLP proteins was analyzed by Western blot using an anti-VP1 primary antibody. “IP” means input.

FIGS. 16A-B provide transmission electron micrographs that illustrate the morphology of the structure of HBcS-GG-VP1 virus-like particles.

FIGS. 17A-B provide transmission electron micrographs that illustrate the morphology of the structure of HBsHBs-GG-HA1 virus-like particles.

FIGS. 18A-F illustrate the neutralization of HBsHBs-GG-HA1 VLP-immunized sera in MDCK cells. An immunofluorescent assay was performed to detect the H1N1-infected MDCK cells after neutralization with various anti-sera (in 1:512 dilution) collected from VLP, HA1, VLP+Alum, and HA1+Alum immunized mice. The 8-week old female Balb/c mice were intraperitoneally immunized with 10 μg of HBsHBs-GG-HA1 VLP (FIGS. 18B, 18D) and an equal mole amount of HA1 protein (4.8 μg; FIGS. 18C, 18E) with or without Alum as adjuvant (FIGS. 18B, 18C are without Alum; FIGS. 18D, 18E are with Alum) at weekly intervals for 4 doses. Sera were collected at day 0 (pre-immune; FIG. 18A) and day 28 post-immunization. Expression of influenza nuclear protein (NP) (green) was detected by FITC-conjugated anti-H1N1 NP antibody in MDCK cells (nuclei labeled with Hoechst in blue). The image data were acquired and quantified by ImageXpress® Micro XL High-Content Image System. Bar=100 μm. “CC” means cell control (FIG. 18F).

FIG. 19 illustrates a graphical representation of the neutralization of HBsHBs-GG-HA1 VLP-immunized sera. The neutralization titer of each serum sample was tested in quadruplicate using an immunofluorescent assay. The immunofluorescent assay was performed to detect the H1N1-infected MDCK cells after neutralization with various antiserum (in 1:512 dilution) collected from VLP, HA1, VLP+Alum, and HA1+Alum immunized mice. The image data was acquired and quantified by ImageExpress® Micro XL High-Content Image System. The preimmune sera did not show any neutralization at 1:8 dilution (the lowest dilution tested) and was therefore shown as a titer of 4 for Geometric Mean Titer (GMT) computation. Each symbol represents a mouse, and the line indicates the GMT of the group. *=P<0.0001.

FIGS. 20A-B illustrate the expression of N-terminally-tagged HBcHBs-GG-HA1 recombinant VLP protein subject to size exclusion chromatography. Protein elution was followed by UV (280 nm). Area (1) of FIG. 20A points to the HBcHBs-GG-HA1 VLPs (void volume), and area (2) points to host cell impurities. The eluate fractions (16-18 and 29-38) were analyzed by Western blot using an anti-influenza H1N1 HA primary antibody (FIG. 20B). IP=mean input; V=void volume; M=monomer fractions.

### DETAILED DESCRIPTION

It should be understood that both the foregoing general description and the following detailed description are exemplary and explanatory but are not restrictive of the invention as claimed. Certain details of one or more embodiments of the invention are set forth in the description below. Other features or advantages of the present invention will be apparent from the non-exhaustive list of representative examples that follows, and also from the appending claims.

As described above, traditional virus-like particle platforms have involved attaching an antigen to a single viral structural protein, often inside a loop structure. But such configurations often hinder the folding and immunogenicity of that antigen. Rather than utilizing the typical monomeric fusion protein design, the present invention comprises a non-monomeric fusion protein design that, surprisingly, permits better antigen folding and enhanced immunogenicity relative to traditional designs, also allowing for optional multivalence, for example, by using one or more immunogenic viral structural proteins.

The present invention is based on several discoveries regarding fusion proteins that are capable of assembling into virus-like particles and that comprise at least two viral structural proteins, at least one antigen or antigenic fragment, and, optionally, one or more linkers. First, the present invention is based on the discovery that said fusion proteins display the antigen or antigenic fragment in a manner that, compared to many other antigen-displaying virus-like particles, better enables it to fold into a conformation that confers immunogenicity. Second, the present invention is based on the discovery that said fusion proteins may optionally comprise viral structural proteins that confer immunogenicity, in at least some instances, independently of immunogenicity conferred by the antigen or antigenic fragment of the fusion protein.

Unless defined otherwise, all technical and scientific terms used herein have the same meanings as are commonly understood by one of skill in the art to which this invention belongs.

As used herein, the articles “a,” “an,” and “any” refer to one or more than one (i.e., at least one) of the grammatical object of the article. For example, “an element” means one element or more than one element.

As used herein, the term “adjuvant” refers to an agent that—if administered to a subject who has been administered, is concurrently administered, or will be administered a composition of the present invention—is capable of contributing to an altered immune response relative to the immune response that would have resulted, had the adjuvant not been administered. Adjuvants are often used to enhance the efficacy of vaccines. This enhancement can occur via adjuvant-dependent changes in: immunomodulation, presentation, targeting, depot generation, induction of cytotoxic T-lymphocyte responses, or a combination thereof. To contribute to an altered immune response, an adjuvant need not necessarily be administered (i) via the same means; (ii) to the same site or target tissue; or (iii) prior to, simultaneously with, or in any other chronological relationship with compositions of the present invention. Types of adjuvants may include, but are not limited to, aluminum salts, bacterial toxins, carbohydrate polymers, cytokines, derivatized polysaccharides, immune-stimulating complexes, lipid A, liposomes, muramyl dipeptide derivatives, nano- and microparticles, non-ionic block copolymers, non-particulate adjuvants, oil-in-water emulsions, particulate adjuvants, saponins, water-in-oil emulsions, or a combination thereof.

As used herein, the term “antigen” refers to a molecule capable of being bound by an antibody or a T-cell receptor (TCR) if presented by MHC molecules. The term “antigen,” as used herein, also encompasses T-cell epitopes. An antigen is additionally capable of one or more of the following: being recognized by the immune system; inducing a humoral immune response; and/or inducing a cellular immune response. However, this may require, at least in some cases, that the antigen comprises or is associated with a T-cell epitope and/or is given in addition to, but not necessarily in any particular chronological relationship with, an adjuvant. An antigen can have one or more epitopes (B- and T-epitopes). The specific reaction referred to above is meant to indicate that the antigen will possibly react, typically in a highly selective manner, with its corresponding antibody or TCR and not with the multitude of other antibodies or TCRs which may be evoked by other antigens. Antigens as used herein may also be mixtures of more than one individual antigen or antigenic fragment.

The term “fusion protein” refers to a protein comprising a non-naturally occurring sequence of amino acids linked by peptide bonds. A fusion protein would not be produced in nature but for the hand of man. As used herein, the term “fusion protein” refers to at least one viral structural protein fused to at least one antigen or antigenic fragment, and, optionally, one or more linkers. For example, a fusion protein may comprise an antigen wherein the N- or C-terminus of the antigen is fused, with or without a linker, to the C- or N-terminus of a viral structural protein. In another example, a fusion protein may comprise a viral structural protein comprising a loop region, wherein the amino acid sequence comprising said loop region has been modified to encode within said loop region one or more antigens or antigenic fragments and, optionally, one or more linkers. In another example, a fusion protein may comprise an antigen, wherein the N-terminus of the antigen is fused, with or without a linker, to an N-terminal viral structural protein, and the C-terminus of the antigen is fused, with or without a linker, to a C-terminal viral structural protein. The viral structural protein components of such fusion proteins, independently or together, may be capable of assembling into macromolecular structures, such as, for example, virus-like particles.

In this application, the term “N-terminal,” when used with respect to a viral structural protein or linker, means that viral structural protein or linker is located N-terminal to the fusion protein's antigen or antigenic fragment. Likewise, the term “C-terminal,” when used with respect to a viral structural protein or linker, means that viral structural protein or linker is located C-terminal to the fusion protein's antigen or antigenic fragment.

As used herein, the term “host cell” refers to single-cell prokaryotic or eukaryotic organisms including but not limited to: actinomycetes, archaea, bacteria, and yeast. A host cell may also be a single cell—including but not limited to cultured cells—from higher-order organisms such as plants and animals, including but not limited to vertebrates such as mammals and invertebrates such as insects.

As used herein, the term “immune response” means at least one of a humoral immune response and cellular immune response leading to the activation or proliferation of at least one of B-lymphocytes, T-lymphocytes, and/or antigen presenting cells. In some instances, the immune response may have low intensity and/or may become detectable only when administering at least one adjuvant in addition to, but not necessarily in any particular chronological relationship with, fusion proteins and/or VLPs of the present invention. “Immunogen” refers to an agent that stimulates the immune system, such that at least one function of the immune system is directly altered by the immunogen. Immunogens may include but are not limited to, for example, immunogenic proteins that elicit at least one of a humoral immune response and a cellular immune response, whether alone or in combination with a carrier and in the presence or absence of an adjuvant. It is possible that an antigen presenting cell may be activated. An immune response is “enhanced” if it is in any way beneficially altered with the administration of the immunogenic agent relative to the immune response without the administration of the agent. For example, amount or type of cytokines secreted or antibodies induced may be altered.

As used herein, the term “linker” refers to at least one amino acid residue that links or otherwise associates the antigen with a viral structural protein. It is possible that the amino acid residues of the linker are composed of naturally occurring amino acids or unnatural amino acids known in the art, all-L or all-D, or a combination thereof. The term “linker” should not be interpreted to mean that the linker exclusively consists of amino acid residues, even if such a linker is comprised by a specific alternative embodiment of the present invention. The present invention also encompasses linkers, without any amino acid residues or with at least one amino acid residue, that comprise a molecule with a sulfhydryl group or cysteine residue. It is possible that such a molecule comprises a C1-C6 alkyl-cycloalkyl (C5, C6), aryl, or heteroaryl-moiety. Association between the linker and at least one of the antigen and the viral structural protein is possibly by way of at least one covalent bond, and possibly by way of at least one peptide bond. In addition, the present invention encompasses flexible linkers, rigid linkers, cleavable linkers, unstructured random coil peptides, or a combination thereof. Flexible linkers may, but do not necessarily, comprise at least one small amino acid, either polar or nonpolar. They may provide for enhanced flexibility or mobility with the associated entities. The incorporation of at least one other amino acid residue, including but not limited to Ser or Thr, helps maintain the stability of the linker under aqueous conditions, perhaps but not necessarily by forming hydrogen bonds with water molecules and reducing unfavorable interaction between the linker and associated entities. Rigid linkers may, but do not necessarily, comprise at least one α-helical structure, Pro-rich sequence, or a combination thereof. They may provide for fixed distance between the associated entities, helping to prevent obstruction, for example, of function. Cleavable linkers may, but do not necessarily, comprise at least one disulfide bond, thrombin-sensitive sequence, protease-sensitive sequence, or a combination thereof. Unstructured random coil peptide linkers may, but do not necessarily, comprise Gly-rich regions, notably unfolded character of any length, or a combination thereof.

As used herein, the term “nucleotide” refers to a monomer comprising a nitrogenous base connected to a sugar phosphate that comprises a sugar, such as ribose or 2′-deoxyribose, connected to one or more phosphate groups. “Polynucleotide” and “nucleic acid” refer to a polymer comprising more than one nucleotide monomer, in which said monomers are often connected by sugar-phosphate linkages of a sugar-phosphate backbone. A polynucleotide need not comprise only one type of nucleotide monomer. For example, the nucleotides comprising a given polynucleotide may be only ribonucleotides, only 2′-deoxyribonucleotides, or a combination of both ribonucleotides and 2′-deoxyribonucleotides. Polynucleotides include naturally occurring nucleic acids, such as deoxyribonucleic acid (“DNA”) and ribonucleic acid (“RNA”), as well as nucleic acid analogs comprising one or more non-naturally occurring monomer. Polynucleotides can be synthesized, for example, using an automated DNA synthesizer. The term “nucleic acid” typically refers to large polynucleotides. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form, but in which “T” replaces “U.” The term “recombinant nucleic acid” refers to a polynucleotide or nucleic acid having sequences that are not naturally joined together. A recombinant nucleic acid may be present in the form of a vector.

As used herein, the term “pathogen” refers to a parasite or other microorganism, or possibly a prion, which is capable of causing an immune response in a living organism. Such parasites and microorganisms may include but are not limited to viruses, bacteria, archaea, protozoa, fungi, algae, rotifers, and helminths. Such prions may include the abnormally folded proteins which are associated with disease states including, but not limited to, transmissible spongiform encephalopathies such as bovine spongiform encephalopathy, Creutzfeldt-Jakob disease, and scrapie.

As used herein, the term “pharmaceutical composition” refers to any formulation wherein the fusion proteins or the virus-like particles of the present invention, or a combination thereof, may be formulated, stored, preserved, altered, administered, or a combination thereof. As described below, the formulation may comprise any pharmaceutically-acceptable diluent, adjuvant, buffer, excipient, carrier, or combination thereof. In general, components of the formulation are selected on the basis of the mode and route of administration, and standard pharmaceutical practice. As used herein, the term “pharmaceutical carrier” refers to any substance or combination thereof with which the fusion proteins or the virus-like particles of the present invention may be physically or chemically mixed, dissolved, suspended, or otherwise combined to yield the pharmaceutical composition of the present invention.

As used herein, the term “pharmaceutically effective amount” refers to an amount capable of or sufficient to maintain or produce a desired physiological result, including but not limited to treating, reducing, eliminating, substantially preventing, or prophylaxing, or a combination thereof, a disease, disorder, or combination thereof. A pharmaceutically effective amount may comprise one or more doses administered sequentially or simultaneously. Those skilled in the art will know to adjust doses of the present invention to account for various types of formulations, including but not limited to slow-release formulations, for the influence of other compositions capable of affecting an immune response, for adjuvants, or for a combination thereof. As used herein, the term “prophylactic” refers to a composition capable of substantially preventing or prophylaxing any aspect of a disease, disorder, or combination thereof. As used herein, the term “therapeutic” refers to a composition capable of treating, reducing, halting the progression of, slowing the progression of, beneficially altering, eliminating, or a combination thereof, any aspect of a disease, disorder, or combination thereof.

As used herein, the term “protein” refers to a molecule that comprises amino acids linearly linked by peptide bonds. This definition of “protein” specifically encompasses polypeptides, oligopeptides, tripeptides, and dipeptides. Proteins may be generated in any manner, including chemical synthesis, and are not necessarily translated from a particular nucleic acid molecule. Proteins include molecules with or without post-expression modifications such as glycosylation, acetylation, and phosphorylation. The term “fragment” refers to a protein comprising an amino acid sequence that is comprised by, but contains fewer residues than, another specified protein. For example, possible fragments of the protein comprising the amino acid sequence Ala-Leu-Gly would be Ala-Leu; Leu-Gly; Ala; Leu; and Gly.

As used herein, the term “subject” refers to any individual to whom administration of the present invention is directed. A subject may be, for example, a mammal. The subject may be a human or veterinary animal, without regard to sex, age, or any combination thereof, and including fetuses. A subject may optionally be afflicted with, at risk for, or a combination thereof a particular disease, disorder, or combination thereof.

As used herein, the term “vaccine” refers to a formulation which comprises one or more fusion proteins of the present invention, VLPs of the present invention, or a combination thereof, in a form capable of administering to a subject, that is capable of affecting a subject's immune response. In a subject, a vaccine may be therapeutic and/or prophylactic for a particular disease, disorder, or combination thereof. Vaccines often, but do not necessarily, comprise a pharmaceutically effective amount of a formulation. Vaccines often, but do not necessarily, affect the immune response in a given subject.

As used herein, the term “vector” refers to the means by which a nucleic acid can be introduced into a host cell to transform the host cell and facilitate expression of the nucleic acid. A vector may comprise a given nucleotide sequence of interest and a regulatory sequence. Vectors may be used for expressing the given nucleotide sequence or maintaining the given nucleotide sequence for replicating it, manipulating it, altering it, truncating it, expanding it, and/or transferring it between different locations (e.g., between different organisms or host cells or a combination thereof).

As used herein, the term “viral structural protein” refers to any protein that contributes to the structure of the capsid or the protein core of a virus, or that otherwise plays a structural role in a viral or virion particle, including but not limited to assembly, folding, or a combination thereof. The term “viral structural protein” encompasses native viral protein sequences, as well as mutants and variants of such native proteins that retain the ability to assemble into a VLP. Viral structural proteins of the present invention may themselves be immunogenic. Viral structural proteins may include envelope or core proteins. For example, Norovirus P, Norovirus S, and HBc are all capsid proteins, whereas HBs is a viral envelope protein. In one embodiment, the viral structural proteins are both viral capsid proteins. In one embodiment, the viral structural proteins are both envelope proteins. In one embodiment, one of the viral structural proteins is a viral capsid protein, and the other is a viral envelope protein

As used herein, the term “virus-like particle” (or “VLP”) refers to a structure resembling a virus particle. A virus-like particle in accordance with the present invention is non-replicating and non-infectious since it lacks all or part of the viral genome, in particular the replicative and infectious components of the viral genome. A virus-like particle in accordance with the present invention may contain nucleic acid residues distinct from the viral genome. Whereas traditional virus-like particles comprise monomeric fusion proteins comprising an antigen and a viral structural protein, virus-like particles of the present invention comprise non-monomeric fusion proteins, such as dimeric fusion proteins, comprising at least one antigen or antigenic fragment, at least two viral structural proteins or fragments thereof, and, optionally, one or more linkers. The fusion proteins comprising a virus-like particle often form a structure with an inherently repetitive organization and, typically, a spherical or tubular shape. One possible embodiment of a virus-like particle in accordance with the present invention is a viral capsid, such as the viral capsid of the corresponding virus, bacteriophage, or RNA-phage. For example, the capsids of RNA-phages or HBcAgs have a spherical form of icosahedral symmetry. The term “capsid-like structure” as used herein, refers to a macromolecular assembly composed of viral structural protein subunits resembling the capsid morphology in the above-defined sense but deviating from typical symmetrical assembly and maintaining a sufficient degree of order and repetitiveness. To form virus-like particles of the present invention, fusion proteins of the present invention may associate via covalent means, noncovalent means, or a combination thereof. Noncovalent associations may comprise, for example, hydrophobic forces, electrostatic forces, pi forces, van der Waals forces, or a combination thereof.

The present invention thus provides a recombinant fusion protein comprising at least one antigen or antigenic fragment, wherein each of the N-terminus and the C-terminus of the antigen or antigenic fragment is fused, with or without a linker, to a viral structural protein. The viral structural proteins are, independently or together, capable of assembling into virus-like particles of the present invention. Virus-like particles of the present invention are capable of displaying antigens or antigenic fragments in conformations that comprise or resemble their native conformation, often in a stable and repetitive manner, thus working as effective vaccines that produce a T- and/or a B-cell-mediated immune response.

As discussed above, the present invention differs from traditional vaccine platforms and even traditional virus-like particle platforms. In particular, the present invention comprises an antigen carried between two viral structural proteins or fragments thereof, with or without linkers, such that the structural proteins remain unaffected or relatively unaffected by the presence of the antigen, and vice versa. This enables the present invention to produce multivalent vaccines and enhanced immunogenicity.

In some embodiments, the antigen in the fusion protein may be a polypeptide from a viral pathogen of mammals, including, but not limited to: enterovirus 71 VP1, influenza virus HA, porcine epidemic diarrhea virus (PEDV), rotavirus VP8, H1N1 M2, H7N9 F, equine herpes virus type 1 glycoprotein 14, Kaposi's sarcoma-associated virus glycoprotein M, human herpes simplex virus type 1 tegument protein, mycobacteriophage 15 predicted 8.2Kd protein, reovirus type 1 sigma-1 protein, sendai virus C′ protein, clover yellow vein virus polyprotein, porcine adenovirus type 3 hexon protein (virion component ii), and human adenovirus type 34 hexon protein.

In other embodiments, the antigen in the fusion protein may be a polypeptide from a bacterial pathogen of mammals, including, but not limited to: Pseudomona species ferredoxin reductase component, Escherichia coli bifunctional penicillin-binding protein, Burkholderia species hydratase/aldolase PhnE, Neisseria meningitidis putative phage virion protein, Methanotroph species methane monooxygenase α-subunit, Synechocystis species exopolyphosphatase gb, Alcaligenes faecalis phenanthrene degradative gene cluster, Synechocystis sp. PCC6803 polyphosphate kinase, Campylobacter jejuni lipopolysaccharide biosynthesis protein wlaK, Acinetobacter species terminal alkane hydroxylase, Herpetosiphon aurantiacus methyltransferase HgiDIM, Mycobacterium tuberculosis hypothetical protein Rv0235c, Mycobacterium tuberculosis hypothetical protein Rv3629c, Streptomyces coelicolor A3(2) anthranilate synthase, Bacillus firmus msyB gene, Escherichia coli URF, Synechocystis sp. PCC6803 cytochrome c oxidase subunit I, Escherichia coli 49 kd protein, and Mycobacterium tuberculosis probable oxidoreductase.

In other embodiments, the antigen in the fusion protein may be a polypeptide from a parasitic pathogen of mammals, including, but not limited to: Plasmodium species HRP II, Plasmodium species pLDH, and Plasmodium species pAldo.

In other embodiments, the antigen in the fusion protein may be a polypeptide from a fungal pathogen of mammals, including, but not limited to Aspergillus versicolor AVS, Aspergillus versicolor AVL, Aspergillus versicolor AveX, Aspergillus flavus Asp fl 1, Aspergillus fumigatus Asp f 1, Aspergillus fumigatus Asp f 2, Aspergillus fumigatus Asp f 3, Aspergillus fumigatus Asp f 4, Aspergillus fumigatus Asp f 5, Aspergillus fumigatus Asp f 6, Aspergillus fumigatus Asp f 7, Aspergillus fumigatus Asp f 8, Aspergillus fumigatus Asp f 9, Aspergillus fumigatus Asp f 10, Aspergillus fumigatus Asp f 11, Aspergillus fumigatus Asp f 12, Aspergillus fumigatus Asp f 13, Aspergillus fumigatus Asp f 15, Aspergillus fumigatus Asp f 16, Aspergillus fumigatus Asp f 17, Aspergillus fumigatus Asp f 18, Aspergillus fumigatus Asp f 25w, Aspergillus fumigatus Asp f 23, Aspergillus fumigatus Asp f 27, Aspergillus fumigatus Asp f 28, Aspergillus fumigatus Asp f 29, Aspergillus niger Asp n 14, Aspergillus niger Asp n 18, Aspergillus niger Asp n 25, Aspergillus niger Asp n, Aspergillus niger Asp o 13, and Aspergillus niger Asp o 21.

In other embodiments, the antigen in the fusion protein may be a polypeptide from a prion, including but not limited to those associated with disease or disorder states including but not limited to transmissible spongiform encephalopathies such as bovine spongiform encephalopathy, Creutzfeldt-Jakob disease, and scrapie.

In some embodiments, at least one of V1 and V2 in the fusion protein may be from a group including, but not limited to: (a) HBc of HBV virus, (b) the small HBV-derived surface antigen (HBsAg), (c) the tS domain of Norovirus capsid protein VP1, (d) the P domain of Norovirus capsid protein VP1, (e) Human Rotavirus VP2, (f) Human Rotavirus VP6, (g) the L1 major capsid protein of human papillomavirus; (h) the VP1 of human polyomavirus, (i) the VP1 of human JC virus, (j) the VP2 of human adeno-associated virus 2, (k) the VP3 of human adeno-associated virus 2, (l) the S and P1 domain of Hepatitis E virus capsid protein VP1, and (m) the P2 domain of Hepatitis E virus capsid protein VP1.

In some embodiments, V1 is linked to the antigen via an N-terminal linker, or V2 is linked to the antigen via a C-terminal linker. In other embodiments, V1 is linked to the antigen via an N-terminal linker and V2 is linked to the antigen via a C-terminal linker, and the N-terminal linker and the C-terminal linker may be the same or different, selected from a group including, but not limited to: the first 59 amino acids from VP1 of EV71; any unstructured random coil peptide of less than 200 amino acids; (GGGGS)ₙ (SEQ ID NO: 2); (Gly)ₙ; (EAAAK)ₙ (SEQ ID NO: 3); A(EAAAK)₄ALEA(EAAAK)₄A (SEQ ID NO: 4); PAPAP (SEQ ID NO: 5); AEAAAKEAAAKA (SEQ ID NO: 6); (X-P)ₙ, where X designates any amino acid, for example, Ala, Lys, or Glu; disulfide; VSQTSKLTRAETVFPDV (SEQ ID NO: 7); PLGLWA (SEQ ID NO: 8); RVLAE (SEQ ID NO: 9); EDVVCCSMSY (SEQ ID NO: 10); GGIEGRGS (SEQ ID NO: 11); TRHRQPRGWE (SEQ ID NO: 12); AGNRVRRSVG (SEQ ID NO: 13); RRRRRRRRR (SEQ ID NO: 14); GFLG (SEQ ID NO: 15); LE; (GS)ₙ; GGSGGHMGSGG (SEQ ID NO: 16); GGSGGGGG (SEQ ID NO: 17); GT; GGSGGSGGSGG (SEQ ID NO: 18); SGGGSSHS (SEQ ID NO: 19); SGGSGGSSHS (SEQ ID NO: 20); SGGSGGSGGSSHS (SEQ ID NO: 21); GGSGG (SEQ ID NO: 22); GGGGSLVPRGSGGGGS (SEQ ID NO: 23); GGGSEGGGSEGGGSEGGG (SEQ ID NO: 24); AAGAATAA (SEQ ID NO: 25); GGGGG (SEQ ID NO: 26); GGSSG (SEQ ID NO: 27); and GSGGGTGGGSG (SEQ ID NO: 28).

Those skilled in the art will understand that at least certain embodiments of the invention involve recombinant nucleic acid techniques including, but not limited to, cloning, polymerase chain reaction, purifying DNA and RNA, restriction enzyme digests, ligations, and expressing recombinant proteins in prokaryotic or eukaryotic cells. Fundamental laboratory techniques for such procedures are adequately described in various well-known publications, such as Michael A. Green, MOLECULAR CLONING: A LABORATORY MANUAL 4^{th} ed. 2012.

For example, the present invention provides recombinant nucleic acids that contain nucleotide sequences that encode fusion proteins of the present invention, which comprise viral structural proteins, at least one antigen or antigenic fragment, and, optionally, one or more linkers. The nucleic acid sequences are operably linked so that they can be transcribed and translated to produce a fusion protein that has the ability to assemble into a VLP.

The nucleic acids that encode fusion proteins of the present invention may comprise either RNA or DNA in many well-known forms, including but not limited to single- or double-stranded entities and vectors. Any of the aforementioned nucleic acids may be constructed using any suitable method known among those well known in the art, as described in, for example, Ralph Rapley, THE NUCLEIC ACID PROTOCOLS HANDBOOK 2000.

Recombinant constructs that encode fusion proteins of the present invention can be prepared in suitable vectors, such as expression vectors, using methods that are conventional and well known in the art. The recombinant construct, such as an expression vector, comprises a nucleic acid which encodes at least one fusion protein of the present invention. The recombinant construct may comprise RNA or DNA in either single- or double-stranded form. Suitable expression vectors for recombinant proteins are conventional and well known in the art. Suitable vectors may, but do not necessarily, comprise, for example: an origin of replication; one or more selectable marker genes; one or more expression control elements, such as a transcriptional control element like a promoter, an enhancer, a terminator, and one or more translation signals; a signal sequence or leader sequence to target, for example, the secretory pathway in a host cell; or a combination thereof. Suitable vectors may, but do not necessarily, comprise one or more detectable markers, such as, for example, a protein that confers resistance to one or more antibiotics. Suitable vectors may be comprised by, but are not necessarily comprised by, a vector expression system, such as a self-replicating nucleic acid.

Any suitable nucleic acid sequences or combinations thereof that encode fusion proteins of the present invention may be used. Nucleic acids can be amplified using suitable methods among those that are well known in the art, such as PCR. One of ordinary skill in the art would know to conduct PCR using primers that are, for example, designed to comprise a lead sequence, a restriction site, and a specified nucleic acid that encodes a protein of interest. After PCR amplification, one of ordinary skill in the art would know to purify the resulting amplicon. One would know how to then separately digest with restriction enzymes the amplicon and expression vector and to then perform a ligation to insert the amplicon into the vector, and to again purify the ligated product. Having generated vectors comprising the insertion sequence encoding a fusion protein of the present invention, one of ordinary skill in the art would know how to then transform or transfect specific host cells and culture said host cells to induce expression. Again, exemplary techniques are described in, for example, Ralph Rapley, THE NUCLEIC ACID PROTOCOLS HANDBOOK 2000.

Methods for producing fusion proteins of the present invention may, but need not necessarily, comprise culturing a host cell that has been transformed or transfected with a recombinant nucleic acid that encodes a fusion protein of the present invention, under conditions suitable for expression of the nucleic acid, and possibly under conditions that are suitable for VLP formation. Such conditions are well known to those of skill in the art. For example, see Kushnir, N., et al., “Virus-like particles as a highly efficient vaccine platform: Diversity of targets and production systems and advances in clinical development,” Vaccine 2012, 31, 58-83, and references therein. Such methods may, optionally, include one or more steps for isolating VLPs, purifying VLPs, or a combination thereof. Such methods may also provide for the production of multivalent VLPs.

The present invention also provides a method to isolate or purify VLPs from host cells, culture media, or a combination thereof. VLPs are possibly isolated or purified directly from conditioned culture media such as the host cell culture media. In addition, host cells can be recovered, host cell homogenate or lysate can be formed, and VLPs can be isolated. Suitable means for lysing cells without destroying VLPs are well known in the art and described in, for example, Kirnbauer, et al., “Efficient self-assembly of human papillomavirus type 16 L1 and L1-L2 into virus-like particles,” J Virol 1993, 67(12):6929-36. Suitable means for isolating VLPs from culture media or host cells are also well known in the art and described in, for example, Wagner, R., et al., “Construction, expression, and Immunogenicity of Chimeric HIV-1 virus-like particles,” Virol 1996, 220, 128-40; Yamschchikov, G. V., et al., “Assembly of SIV virus-like particles containing envelope proteins using a baculovirus expression system,” Virol 1995, 214, 50-58; Sakuragi, S., et al., “HIV type 1 Gag virus-like particle budding from spheroplasts of Saccharomyces cerevisiae,” PNAS 2002, 99, 7956-61; Andreadis, S. T., et. al., “Large-scale processing of recombinant retroviruses for gene therapy,” Biotechnol Prog 1999, 15, 1-11; Bachmann, A. S., et al., “A simple method for the rapid purification of copia virus-like particles from Drosophila Schneider 2 cells,” J. Virol. Methods 2004, 115, 159-65. Such means may include density gradient centrifugation such as sucrose gradients, pelleting, and PEG-precipitation, and they may also include standard purification techniques such as ion exchange and gel filtration chromatography.

Centrifugation on a sucrose gradient or cushion is one possible means of isolating VLPs from cellular components, and at least one study indicates that after ultracentrifigation, unassembled proteins become concentrated in the upper fractions, which have relatively low sucrose concentration, whereas assembled VLPs become concentrated in the lower fractions, which have relatively high sucrose concentration. See, for example, Zlotnick, A., et al., “Separation and crystallization of T=3 and T=4 icosahedral complexes of the hepatitis B virus core protein,” Acta Cryst 1999, D 55:717-20. The aforementioned techniques may be useful individually, in succession, or when incorporated into a larger system.

Although not necessary to practice the present invention, electron microscopy provides a means for confirming VLP assembly. For example, after ultracentrifugation, a sample from the lower fraction, which presumably contains assembled VLPs, can be inspected via EM. Suitable techniques are well known to those skilled in the art, as in, for example, Han, M. G., et al., “Self-assembly of the recombinant capsid protein of a bovine norovirus (BoNV) into virus-like particles and evaluation of cross-reactivity of BoNV with human noroviruses,” J Clin Microbiol 2005, 43(2):778-85.

Confirmation of antigenicity may be achieved by administering the fusion proteins and/or virus-like particles of the invention to rodents prior to analyzing blood serum for neutralization activity. See, for example, Xu L, et al., “Protection against Lethal Enterovirus 71 Challenge in Mice by a Recombinant Vaccine Candidate Containing a Broadly Cross-Neutralizing Epitope within the VP2 EF Loop,” Theranostics 2014; 4(5):498-513. Alternatively, neutralization can be measured by blue native PAGE. Suitable BN-PAGE techniques are well known to those skilled in the art, as in, for example, Moore, P. L., et al., “Nature of nonfunctional envelope proteins on the surface of human immunodeficiency virus type 1,” J Virol 2006, 80, 2515-28. Such tests of antigenicity may also comprise an in vitro comparison of the immunogenicity between traditional VLP platforms and those of the present invention.

Fusion proteins of the present invention, VLPs of the present invention, or a combination thereof, may be administered through any suitable means, enterally, parenterally, or otherwise, and including, but not limited to, buccally, intradermally, intramuscularly, intraperitoneally, intravenously, intravesically, intrathecally, ocularly, orally, rectally, subcutaneously, sublingually, topically, or a combination thereof, sequentially or simultaneously.

Formulations suitable for administration of the present invention may comprise, possibly among other things well known to those of skill in the art: aqueous and non-aqueous solutions; antioxidants; bacteriostats; buffers; solutes that affect isotonicity; preservatives; solubilizers; stabilizers; suspending agents; thickening agents; or a combination thereof.

In addition or in the alternative, formulations suitable for administration of the present invention may comprise, possibly among other things well known to those of skill in the art: gels; PEG such as PEG 400; propylene glycol; saline; sachets; water; other appropriate liquids known in the art; or a combination thereof.

Also in the addition or in the alternative, formulations suitable for administration of the present invention may comprise, possibly among other things well known to those of skill in the art: binders; buffering agents; calcium phosphates; cellulose; colloids, such as colloidal silicon dioxide; colorants; diluents; disintegrating agents; dyes; fillers; flavoring agents; gelatin; lactose; magnesium stearate; mannitol; microcrystalline gelatin; moistening agents; paraffin hydrocarbons; pastilles; polyethylene glycols; preservatives; sorbitol; starch, such as corn starch, potato starch, or a combination thereof; stearic acid; sucrose; talc; triglycerides; or a combination thereof.

Also in addition or in the alternative, formulations suitable for administration of the present invention may comprise, possibly among other things well known to those of skill in the art: alcohol such as benzyl alcohol or ethanol; benzalkonium chloride; buffers such as phosphate buffers, acetate buffers, citrate buffers, or a combination thereof; carboxymethylcellulose or microcrystalline cellulose; cholesterol; dextrose; juice such as grapefruit juice; milk; phospholipids such as lecithin; oil such as vegetable, fish, or mineral oil, or a combination thereof; other pharmaceutically compatible carriers known in the art; or a combination thereof.

Also in the addition or in the alternative, formulations suitable for administration of the present invention may comprise, possibly among other things well known to those of skill in the art: biodegradables such as poly-lactic-coglycolic acid (PLGA) polymer, other entities whose degradation products can quickly be cleared from a biological system, or a combination thereof.

Formulation degradability—for example, in sustained-release formulations—can be adjusted by techniques known to those skilled in the art. See, for example, Danny Lewis, CONTROLLED RELEASE OF BIOACTIVE AGENTS FROM LACTIDE/GLYCOLIDE POLYMERS, IN BIODEGRADABLE POLYMERS AS DRUG DELIVERY SYSTEMS, Chasin, M. and Langer, R., eds. 1990. Marcel Dekker: New York.

Formulations of the present invention may be administered in unit-dose form, multi-dose form, or a combination thereof. They may be packaged in unit-dose containers, multi-dose containers, or a combination thereof. The present invention may exist in ampoules; cachets; capsules; granules; lozenges; powders; tablets; vials; emulsions, including but not limited to acacia emulsions; suspensions; or a combination thereof.

An immunogenic composition may comprise fusion proteins of the present invention, VLPs of the present invention, nucleic acids encoding fusion proteins of the claimed invention, or combinations thereof. One or more such compositions, identical, different, or a combination thereof, may be administered using the same or different formulations. Such immunogenic compositions may be administered prophylactically, therapeutically, or a combination thereof, and may be administered one or more times to a given subject. For example, multiple administrations to a given subject might comprise a priming administration followed by booster administrations to test or optimize the desired immune response or lack thereof. Repeated administration to a given subject need not necessarily comprise the same immunogenic composition. Immunogenic compositions may comprise, but need not necessarily comprise, a suitable nucleic acid delivery system, such as, but not limited to, emulsions, particles, vectors, viral particles, liposomes, lipoplexes, replicons, or combinations thereof.

Fusion proteins of the present invention, VLPs of the present invention, and combinations thereof may optionally be administered sequentially or simultaneously with one or more adjuvants, as discussed above. Adjuvants may modify cytokine activity, for example, through broad upregulation of the whole immune system, through upregulation of specific cytokines, through downregulation of specific cytokines, or any combination thereof. Alternatively or in addition, adjuvants may facilitate presentation, to immune effector cells, of a given antigen or antigenic fragment of the present invention in a conformation comprising or resembling its native conformation. Alternatively or in addition, adjuvants may facilitate delivery of antigens or antigenic fragments of the present invention to immune effector cells. Alternatively or in addition, adjuvants may trap a given antigen or antigenic fragment of the present invention in an injection site, possibly, for example, to assist extended delivery, to prevent degradation, or a combination thereof. Alternatively or in addition, adjuvants may facilitate induction of C8+ cytotoxic T-lymphocyte responses.

Adjuvants, if optionally used, may be selected from a group including but not limited to: aluminum hydroxide; aluminum phosphate; alum; microdroplets of water stabilized by a surfactant, such as mannide monooleate, in a continuous oil phase, such as mineral oil, squalene, or squalane; Freund's incomplete adjuvant; microdroplets of oil, such as squalene or squalane, stabilized by surfactants, such as Tween 80 or Span 85, in a continuous water phase; immune-stimulating complexes; liposomes; nano- and microparticles; particulate adjuvants such as calcium salts, proteasomes, virosomes, stearyl tyrosine, gamma-inulin, algammulin, non-particulate adjuvants; muramyl dipeptide and derivatives thereof, including N-acetyl muramyl-L-alanyl-D-isoglutamine, threonyl MDP, murabutide, N-acetylglucosaminyl-MDP, GMDP, murametide and nor-MDP, and MTP-PE; non-ionic block copolymers, such as CRL 1005; saponins, including mixtures of triterpenoids, Saponin, Quil A, Spikoside, QS21, and ISCOPREP™ 703; Lipid A; 4′ monophosphoryl lipid A (MPL); cell wall skeleton; cytokines; carbohydrate polymers such as mannan, glucan, acemannan, lentinan; derivatized polysaccharides, including dextrins, diethylaminoethyl dextran; bacterial toxins, including cholera toxin, CTB pentamer, E. coli labile toxin, LTB, or mutants or other derivatives thereof; other non-particulate adjuvants such as dehydroepiandrosterone, Vitamin D3, trehalose dimycolate, P₃CSS, Poly I:C, Poly ICLC, Poly A:U, or a combination thereof.

All publications and other references mentioned herein are herein fully incorporated by reference for the purpose of disclosing and describing methods and compositions which might be used in making or using the present invention.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

In the examples below, abbreviations not defined have their generally accepted meanings, and the following abbreviations have the following meanings:

HBc and H refer to the subdomain from the core antigen of Hepatitis B virus.

S is the S domain of the Norovirus VP1 protein.

P is the P domain of the Norovirus VP1 protein.

EV71 is enterovirus type 71.

VP1 is the capsid protein of EV71.

VP8 is the VP8 domain from Rotavirus.

M2 is the transmembrane protein of H1N1 virus.

M2e is the extracellular domain of M2.

HA1 is the influenza A hemagglutinin 1 protein.

CVB3 VP1 is the coxsackievirus B3 VP1 capsid protein.

Sp₁ is the S domain+the P1 domain of the Hepatitis E virus.

P₂ is the P domain of the Hepatitis E virus.

HBs is the small HBV-derived surface antigen.

G is a flexible linker having the following sequence: (GGGGS)₃ (SEQ ID NO: 29) (i.e., GGGGSGGGGSGGGGS) (SEQ ID NO: 29).

E is a rigid linker having the following sequence (EAAAK)₃ (SEQ ID NO: 30) (i.e., EAAAKEAAAKEAAAK) (SEQ ID NO: 30).

R is a random-coil linker having the amino acid sequence of the first 59 amino acids (amino acids 1-59) of the EV71 VP1 protein (i.e., GDRVADVIESSIGDSVSRALTHALPAPTGQNTQVSSHRLDTGKVPALQAAEI GASSNAS) (SEQ ID NO: 31).

The virus-like-particles (VLPs) described in these Examples are named as follows: V1V2-L1L2-Ag. For example, the VLP named SP-GG-VP1 contains an N-terminal SP viral structural protein, an N-terminal (GGGGS)₃ flexible linker (SEQ ID NO: 29), a VP1 antigen, a C-terminal (GGGGS)₃ flexible linker (SEQ ID NO: 29), and a C-terminal SP viral structural protein.

### Example 1

### VLP Template Construction

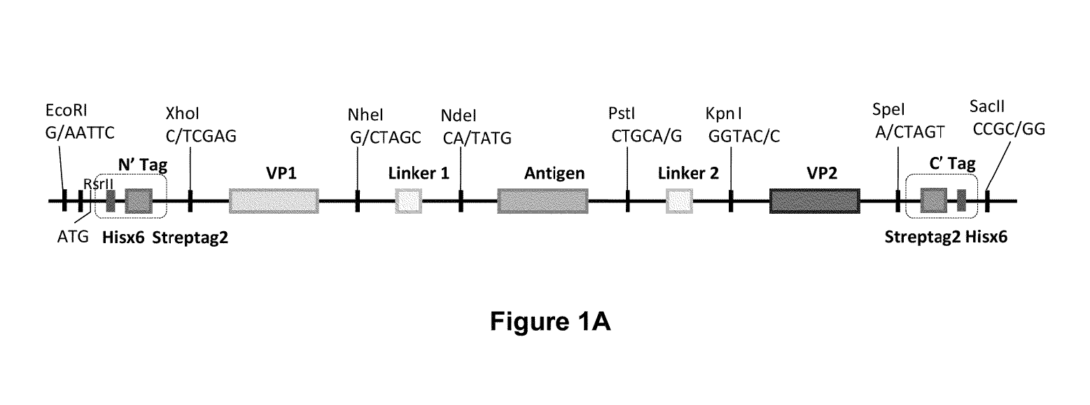

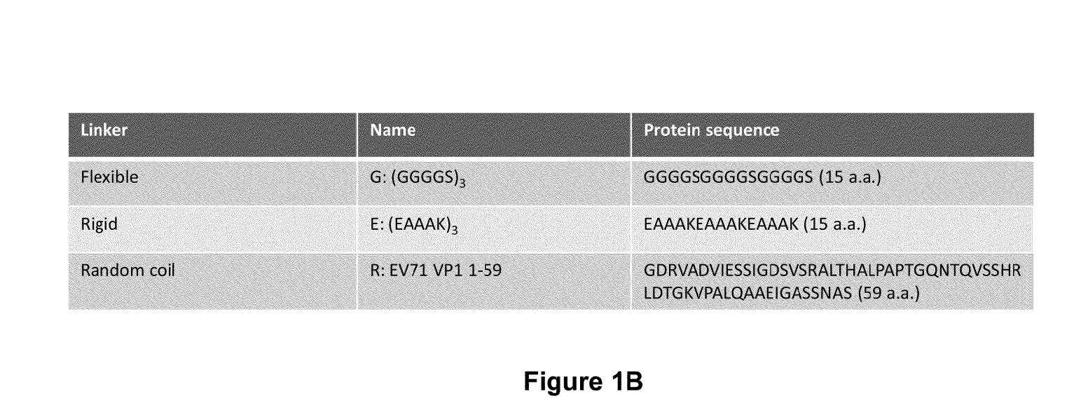

The VLP template (FIG. 1) was synthesized by GenScript in the plasmid pUC57 using a codon optimization specific for yeast. The fragment of the VLP template was subcloned into the yeast expression plasmid vector pPICZ A using EcoRI (5′-end) and SacII (3′-end) sites and expression was regulated using the methanol inducible AOX1 promoter. Sequences encoding the N-terminal virus structural protein (VP1) were cloned into the template using a XhoI+NheI pair of restriction sites at the 5′ and 3′ ends. Sequences encoding the N-terminal linker (Linker 1) were cloned into the template using a NheI+NdeI pair of restriction sites at the 5′ and 3′ ends. Sequences encoding the antigen were cloned into the template using a NdeI+PstI pair of restriction sites at the 5′ and 3′ ends. Sequences encoding the C-terminal linker (Linker 2) were cloned into the template using a PstI+KpnI pair of restriction sites at the 5′ and 3′ ends. Sequences encoding the C-terminal viral structural protein (VP2) were cloned into the template using a KpnI+SpeI pair of restriction sites at the 5′ and 3′ ends.

For example, in the VLP named SP-GG-VP1, the S domain of the Norovirus VP1 protein was cloned into the template using XhoI+NheI, the N-terminal flexible linker (GGGGS)₃ (SEQ ID NO: 29) was cloned into the template using NheI+NdeI, the VP1 antigen was cloned into the template using NdeI+PstI, the C-terminal flexible linker (GGGGS)₃ (SEQ ID NO: 29) was cloned into the template using PstI+KpnI, and the P domain of the Norovirus VP1 protein was cloned into the template using KpnI+SpeI.

The following recombinant proteins of Examples 1-1 through 1-108 were generated using the VLP template:

| Example | Name | VP1 | L1 | Ag | L2 | VP2 |
|---|---|---|---|---|---|---|
| 1-1 | SP-GG | S | G | — | G | P |
| 1-2 | SP-GE | S | G | — | E | P |
| 1-3 | SP-GR | S | G | — | R | P |
| 1-4 | SP-EG | S | E | — | G | P |
| 1-5 | SP-EE | S | E | — | E | P |
| 1-6 | SP-ER | S | E | — | R | P |
| 1-7 | SP-RG | S | R | — | G | P |
| 1-8 | SP-RE | S | R | — | E | P |
| 1-9 | SP-RR | S | R | — | R | P |
| 1-10 | SP-GG-VP1 | S | G | VP1 | G | P |
| 1-11 | SP-GE-VP1 | S | G | VP1 | E | P |
| 1-12 | SP-GR-VP1 | S | G | VP1 | R | P |
| 1-13 | SP-EG-VP1 | S | E | VP1 | G | P |
| 1-14 | SP-EE-VP1 | S | E | VP1 | E | P |
| 1-15 | SP-ER-VP1 | S | E | VP1 | R | P |
| 1-16 | SP-RG-VP1 | S | R | VP1 | G | P |
| 1-17 | SP-RE-VP1 | S | R | VP1 | E | P |
| 1-18 | SP-RR-VP1 | S | R | VP1 | R | P |
| 1-19 | SP-GE-HA1 | S | G | HA1 | E | P |
| 1-20 | SP-GG-HA1 | S | G | HA1 | G | P |
| 1-21 | SP-GR-HA1 | S | G | HA1 | R | P |
| 1-22 | SP-EE-HA1 | S | E | HA1 | E | P |
| 1-23 | SP-EG-HA1 | S | E | HA1 | G | P |
| 1-24 | SP-ER-HA1 | S | E | HA1 | R | P |
| 1-25 | SP-RE-HA1 | S | R | HA1 | E | P |
| 1-26 | SP-RG-HA1 | S | R | HA1 | G | P |
| 1-27 | SP-RR-HA1 | S | R | HA1 | R | P |
| 1-28 | SP-GG-M2e | S | G | M2e | G | P |
| 1-29 | SP-GE-M2e | S | G | M2e | E | P |
| 1-30 | SP-GR-M2e | S | G | M2e | R | P |
| 1-31 | SP-EG-M2e | S | E | M2e | G | P |
| 1-32 | SP-EE-M2e | S | E | M2e | E | P |
| 1-33 | SP-ER-M2e | S | E | M2e | R | P |
| 1-34 | SP-RG-M2e | S | R | M2e | G | P |
| 1-35 | SP-RE-M2e | S | R | M2e | E | P |
| 1-36 | SP-RR-M2e | S | R | M2e | R | P |
| 1-37 | SH-GG-VP1 | S | G | VP1 | G | H |
| 1-38 | SH-GE-VP1 | S | G | VP1 | E | H |
| 1-39 | SH-GR-VP1 | S | G | VP1 | R | H |
| 1-40 | SH-EG-VP1 | S | E | VP1 | G | H |
| 1-41 | SH-EE-VP1 | S | E | VP1 | E | H |
| 1-42 | SH-ER-VP1 | S | E | VP1 | R | H |
| 1-43 | SH-RG-VP1 | S | R | VP1 | G | H |
| 1-44 | SH-RE-VP1 | S | R | VP1 | E | H |
| 1-45 | SH-RR-VP1 | S | R | VP1 | R | H |
| 1-46 | SP-GG-VP8 | S | G | VP8 | G | P |
| 1-47 | SP-GE-VP8 | S | G | VP8 | E | P |
| 1-48 | SP-GR-VP8 | S | G | VP8 | R | P |
| 1-49 | SP-EG-VP8 | S | E | VP8 | G | P |
| 1-50 | SP-EE-VP8 | S | E | VP8 | E | P |
| 1-51 | SP-ER-VP8 | S | E | VP8 | R | P |
| 1-52 | SP-RG-VP8 | S | R | VP8 | G | P |
| 1-53 | SP-RE-VP8 | S | R | VP8 | E | P |
| 1-54 | SP-RR-VP8 | S | R | VP8 | R | P |
| 1-55 | SP-GG-CVB3 VP1 | S | G | CVB3 VP1 | G | P |
| 1-56 | SP-GE-CVB3 VP1 | S | G | CVB3 VP1 | E | P |
| 1-57 | SP-GR-CVB3 VP1 | S | G | CVB3 VP1 | R | P |
| 1-58 | SP-EG-CVB3 VP1 | S | E | CVB3 VP1 | G | P |
| 1-59 | SP-EE-CVB3 VP1 | S | E | CVB3 VP1 | E | P |
| 1-60 | SP-ER-CVB3 VP1 | S | E | CVB3 VP1 | R | P |
| 1-61 | SP-RG-CVB3 VP1 | S | R | CVB3 VP1 | G | P |
| 1-62 | SP-RE-CVB3 VP1 | S | R | CVB3 VP1 | E | P |
| 1-63 | SP-RR-CVB3 VP1 | S | R | CVB3 VP1 | R | P |
| 1-64 | SHBc-GG-HA1 | S | G | HA1 | G | HBc |
| 1-65 | SHBc-GE-HA1 | S | G | HA1 | E | HBc |
| 1-66 | SHBc-GR-HA1 | S | G | HA1 | R | HBc |
| 1-67 | SHBc-EG-HA1 | S | E | HA1 | G | HBc |
| 1-68 | SHBc-EE-HA1 | S | E | HA1 | E | HBc |
| 1-69 | SHBc-ER-HA1 | S | E | HA1 | R | HBc |
| 1-70 | SHBc-RG-HA1 | S | R | HA1 | G | HBc |
| 1-71 | SHBc-RE-HA1 | S | R | HA1 | E | HBc |
| 1-72 | SHBc-RR-HA1 | S | R | HA1 | R | HBc |
| 1-73 | SP₁P₂-GG-VP1 | SP₁ | G | VP1 | G | P₂ |
| 1-74 | SP₁P₂-GE-VP1 | SP₁ | G | VP1 | E | P₂ |
| 1-75 | SP₁P₂-GR-VP1 | SP₁ | G | VP1 | R | P₂ |
| 1-76 | SP₁P₂-EG-VP1 | SP₁ | E | VP1 | G | P₂ |
| 1-77 | SP₁P₂-EE-VP1 | SP₁ | E | VP1 | E | P₂ |
| 1-78 | SP₁P₂-ER-VP1 | SP₁ | E | VP1 | R | P₂ |
| 1-79 | SP₁P₂-RG-VP1 | SP₁ | R | VP1 | G | P₂ |
| 1-80 | SP₁P₂-RE-VP1 | SP₁ | R | VP1 | E | P₂ |
| 1-81 | SP₁P₂-RR-VP1 | SP₁ | R | VP1 | R | P₂ |
| 1-82 | SP₁P₂-GG-HA1 | SP₁ | G | HA1 | G | P₂ |
| 1-83 | SP₁P₂-GE-HA1 | SP₁ | G | HA1 | E | P₂ |
| 1-84 | SP₁P₂-GR-HA1 | SP₁ | G | HA1 | R | P₂ |
| 1-85 | SP₁P₂-EG-HA1 | SP₁ | E | HA1 | G | P₂ |
| 1-86 | SP₁P₂-EE-HA1 | SP₁ | E | HA1 | E | P₂ |
| 1-87 | SP₁P₂-ER-HA1 | SP₁ | E | HA1 | R | P₂ |
| 1-88 | SP₁P₂-RG-HA1 | SP₁ | R | HA1 | G | P₂ |
| 1-89 | SP₁P₂-RE-HA1 | SP₁ | R | HA1 | E | P₂ |
| 1-90 | SP₁P₂-RR-HA1 | SP₁ | R | HA1 | R | P₂ |
| 1-91 | SP₁P-GG-VP1 | SP₁ | G | VP1 | G | P |
| 1-92 | SP₁P-GE-VP1 | SP₁ | G | VP1 | E | P |
| 1-93 | SP₁P-GR-VP1 | SP₁ | G | VP1 | R | P |
| 1-94 | SP₁P-EG-VP1 | SP₁ | E | VP1 | G | P |
| 1-95 | SP₁P-EE-VP1 | SP₁ | E | VP1 | E | P |
| 1-96 | SP₁P-ER-VP1 | SP₁ | E | VP1 | R | P |
| 1-97 | SP₁P-RG-VP1 | SP₁ | R | VP1 | G | P |
| 1-98 | SP₁P-RE-VP1 | SP₁ | R | VP1 | E | P |
| 1-99 | SP₁P-RR-VP1 | SP₁ | R | VP1 | R | P |
| 1-100 | SP₁P-GG-HA1 | SP₁ | G | HA1 | G | P |
| 1-101 | SP₁P-GE-HA1 | SP₁ | G | HA1 | E | P |
| 1-102 | SP₁P-GR-HA1 | SP₁ | G | HA1 | R | P |
| 1-103 | SP₁P-EG-HA1 | SP₁ | E | HA1 | G | P |
| 1-104 | SP₁P-EE-HA1 | SP₁ | E | HA1 | E | P |
| 1-105 | SP₁P-ER-HA1 | SP₁ | E | HA1 | R | P |
| 1-106 | SP₁P-RG-HA1 | SP₁ | R | HA1 | G | P |
| 1-107 | SP₁P-RE-HA1 | SP₁ | R | HA1 | E | P |
| 1-108 | SP₁P-RR-HA1 | SP₁ | R | HA1 | R | P |
| 1-109 | S-VP1-S | S | G | VP1 | G | P |

### Example 1A

### S-VP1-S Construction

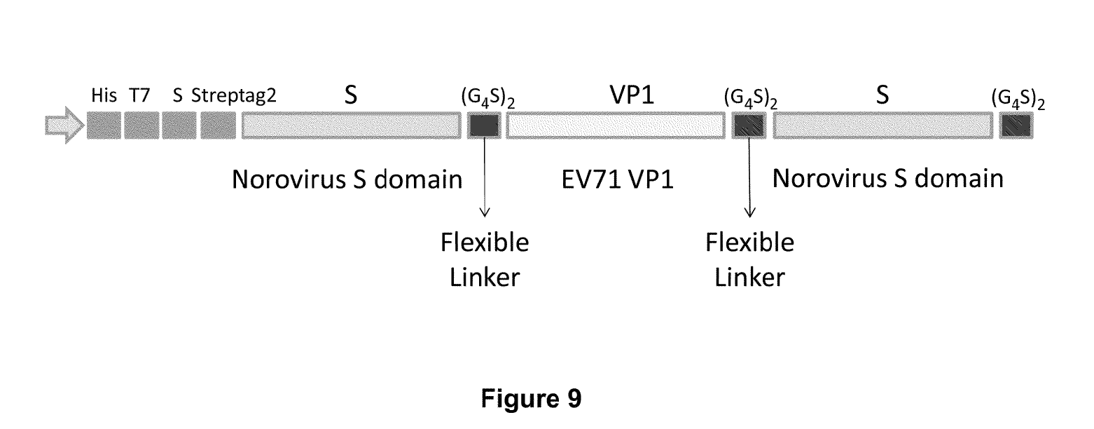

The coding region of S-VP1-S (see FIG. 9) was synthesized by GenScript in the plasmid pUC57, with codon optimization for E. coli. The fragment of the VLP template was subcloned into plasmid vector pCRT7NT (Invitrogen) by PCR and expression was regulated by the IPTG-inducible T7 promoter. This technique was used to produce the recombinant protein of Example 1-109 in the above table.

### Example 2

### Yeast Transformation

Recombinant plasmid DNA was linearized with PmeI (NEB) and clean-up by NucleoSpin® (Macherey-Nagel) for subsequent transformation. 5-10 μg of linearized plasmid DNA was transformed into Pichia pastoris host strain GS115 by the lithium chloride method according to the instruction manual of the EasySelect™ Pichia Expression kit (Invitrogen). The transformants were plated on YPDS plates (1% (w/v) yeast extract, 2% (w/v) peptone, 2% (w/v) dextrose, and 1.5% (w/v) agar) containing 50 μg/ml Zeocin (Invivogen). Zeocin-resistant clones were selected and the insertion was confirmed by colony PCR using the following primers: 5′ AOX1 primer: 5″-GACTGGTTCCAATTGACAAGC-3″ (SEQ ID NO: 32); 3′ AOX1 primer: 5″-GCAAATGGCATTCTGACATCC-3″ (SEQ ID NO: 33).

### Example 3

### Protein Expression

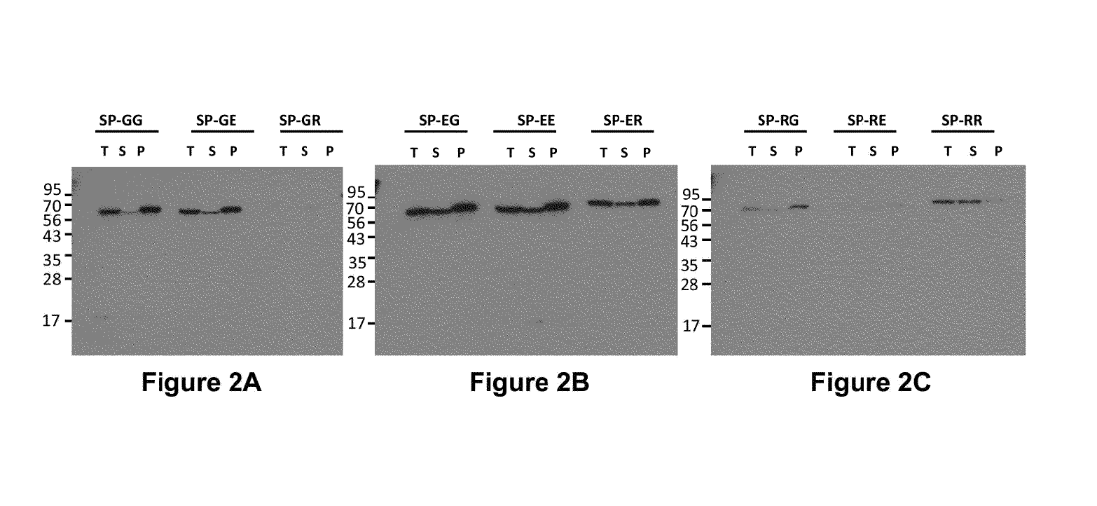

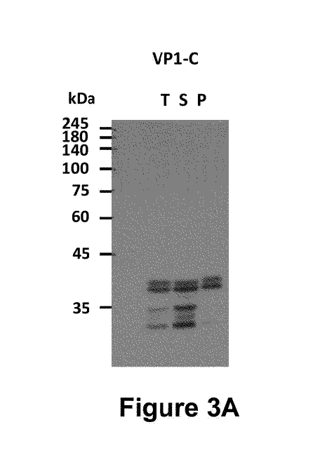

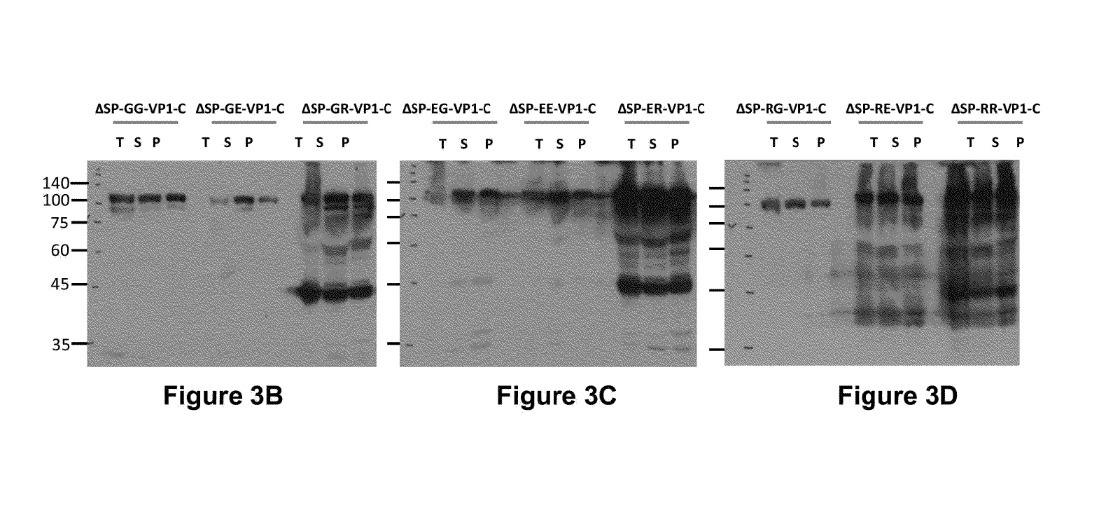

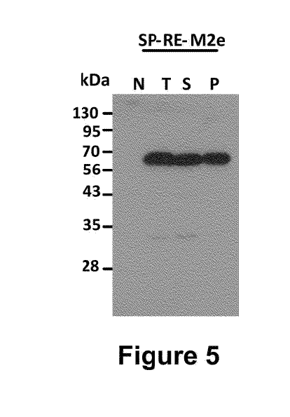

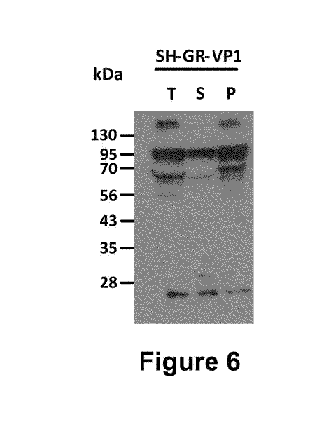

The recombinant protein expression for all but the S-VP1-S construct was induced by methanol induction. Single colonies were incubated in 40 ml of YPD medium (1% (w/v) yeast extract, 2% (w/v) peptone, and 2% (w/v) dextrose) in 250 ml flasks. The cultures were grown at 30° C. in an orbital-sharking incubator (250 rpm) until the cells were in log-phase growth (OD₆₀₀=1.3-1.8). The cells were harvested by centrifuging at 1500×g for 5 minutes at room temperature (RT). The supernatant was decanted and the cell pellets were resuspended to an OD₆₀₀ of 1.0 in YP medium (1% (w/v) yeast extract and 2% (w/v) peptone) with 0.5% (v/v) methanol. After 24 hours, the cell pellets were collected by centrifuging and stored at −80° C. until ready to assay. Cell pellets were resuspended in cold KCl buffer (100 mM KCl, 20 mM HEPES, 1 mM EDTA, and 1 mM PMSF, pH8.0) and then homogenized by French press (20,000 psi; EmulsiFlex-B15, AVESTIN). The soluble and insoluble recombinant VLP proteins were separated by centrifuging (15000×g for 30 minutes at 4° C.) and analyzed by Western blot (FIGS. 2-6).

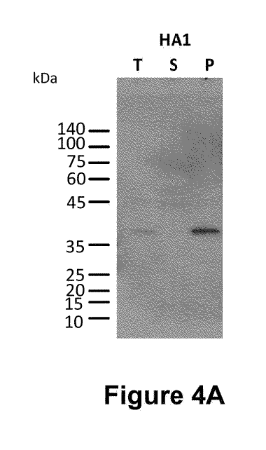

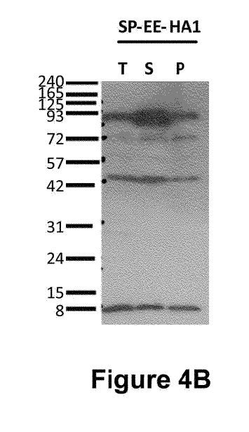

As shown in FIG. 4, the HA1 antigen expressed alone is mostly insoluble (FIG. 4A). But once the HA1 antigen is inserted into a VLP construct of the invention, solubility is improved (FIG. 4B). This indicates that the VLP design of the invention assists HA1 antigen folding.

### Example 3A

### S-VP1-S Protein Expression

Recombinant protein expression of S-VP1-S was induced by 1 mM IPTG induction. A single colony was incubated in 10 mL of LB medium (1% (w/v) Tryptone, 0.5% (w/v) yeast extract, and 1% (w/v) sodium chloride) in a 250 mL flask. The culture was grown at 37° C. in an orbital-shaking incubator (250 rpm) until the cells were in log-phase growth (OD₆₀₀=0.6-0.8). The cells were harvested by centrifugation at 8000×g for 10 minutes at room temperature. The cell pellets were collected by centrifugation and stored at −80° C. until ready to assay. Cell pellets were resuspended in cold KCl buffer (100 mM KCl, 20 mM HEPES, 1 mM EDTA, and 1 mM PMSF, pH 8.0) and then homogenized by French press (20,000 psi; EmulsiFlex-B15, AVESTIN). The soluble and insoluble recombinant VLP proteins were separated by centrifugation (15,000×g for 30 min at 4°) and analyzed by Western blot (data not shown).

### Example 4

### VLP Purification and Characterization

VLP formation was characterized by sucrose density gradient and size-exclusion chromatography. Yeast cells were collected and resuspended in cold KCl buffer and then homogenized by French press. The supernatant was collected by centrifugation (15000×g for 30 minutes at 4° C.) and used as the crude extract for size-exclusion chromatography (Superose 6 Increase 10/300 GL, GE healthcare) and 10%-50% continuous sucrose gradient ultracentrifugation (35,000 rpm for 4 h; SW 41 Ti rotor, Optima™ L-100 XP, Beckman). VLP formation was determined based on the appearance of recombinant VLP proteins in the void volume of size-exclusion chromatography and in the 20%-40% fractions of the sucrose gradient by Western blot analysis.

For purification of the SP-GG-VP1 VLPs, VLPs in the crude extract were purified by Nickel-column chromatography (Ni Sepharose™ 6 Fast Flow, GE healthcare) and the proteins were assayed by Coomassie blue-stained SDS-polyacrylamide gel electrophoresis and Western blot.

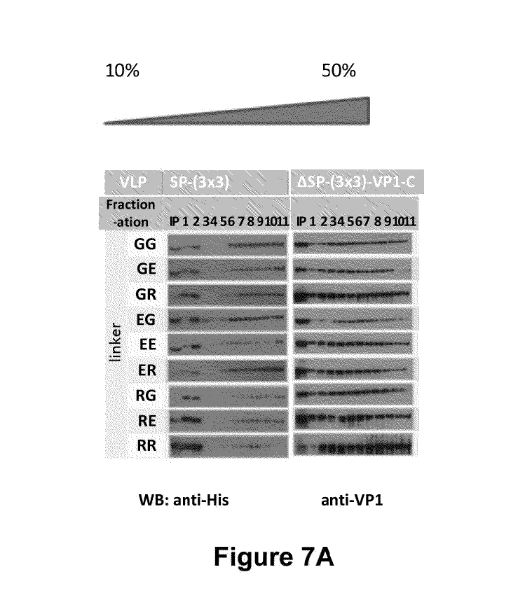

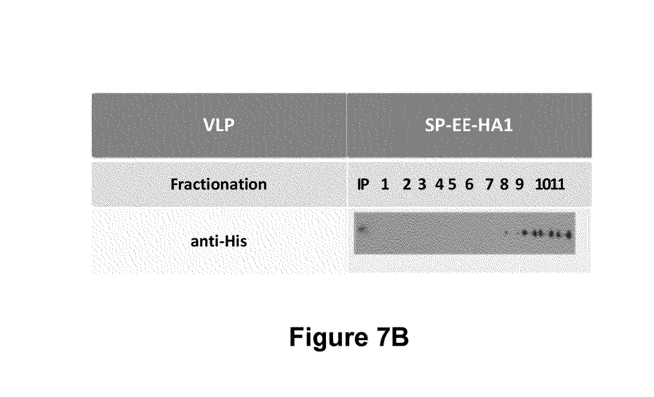

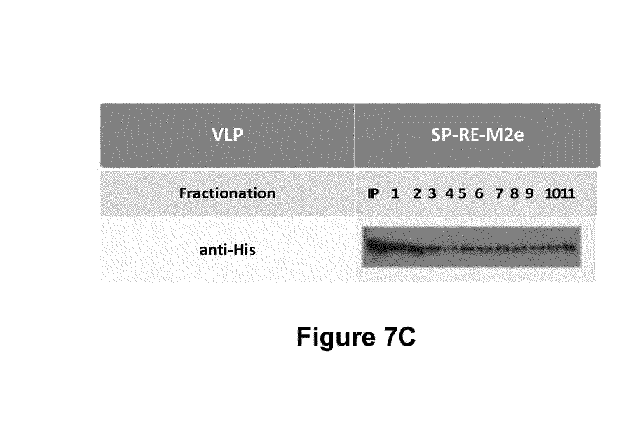

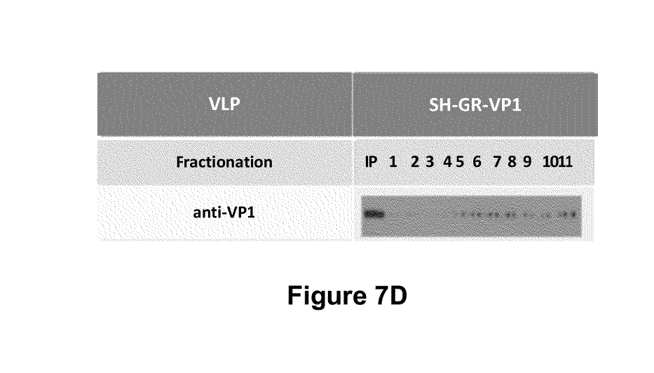

The results of a sucrose gradient analysis of several VLP constructs of the invention are depicted in FIG. 7.

### Example 5

### Transmission Electron Microscopy (TEM)

The particle size and morphology of the VLPs were characterized by TEM. Purified VLPs were adsorbed onto formvar/carbon-coated copper grids (Electron Microscope Science) and negative stained with 1.5% aqueous uranyl acetate. The samples were imaged using JEOL JEM-1200EX II Transmission Electron Microscope.

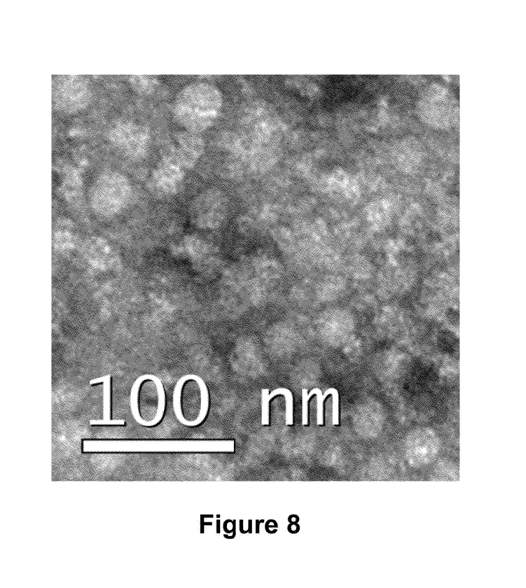

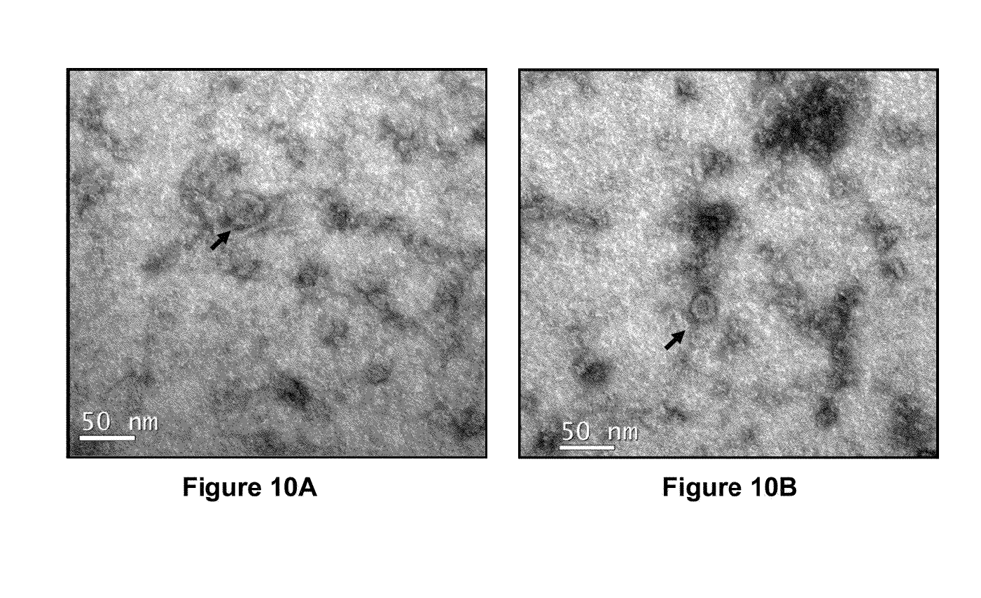

Electron micrographs of SP-GG-VP1 and S-VP1-S VLPs are shown in FIGS. 8 and 10, respectively.

### Example 6

### Cell Lines and Virus Strains

Human rhabdomyosarcoma (RD) cells were passaged in Dulbecco's Modified Eagle's Medium-high glucose (DMEM-HG, Caisson) containing 10% FBS (Genedirex), 1% L-glutamine (Caisson) and 1% penicillin/streptomycin (Caisson) in a humidified atmosphere at 37° C. and 5% CO₂. The EV71 virus (B5 genotype) was obtained from Taiwan CDC (CDC#2013-EV-00017) and propagated in RD cells with 2% FBS at MOI 0.01. The virus stocks were collected from the supernatants harvested at three days post infection. To estimate viral infectivity titers, EV71 was diluted 10-fold and incubated with RD cells on a 96-well plate. CPE was observed using an inverted microscope after an incubation period of 4 days. The 50% tissue culture infectious doses (TCID50) of EV71 were calculated by the method of Reed, L. J. and Muench, H. (1938) “A simple method of estimating fifty percent endpoints” The American Journal of Hygiene 27: 493-497.

MDCK cells were passaged in DMEM supplemented with 10% FBS, 1% L-glutamine, and 1% penicillin/streptomycin in a humidified atmosphere at 37° C. and 5% CO₂. The H1N1 virus (A/Taiwan/80813/2013) whicwash obtained from Taiwan CDC and was propagated in MDCK cells with serum free DMEM supplemented with 1 ug/mL TPCK-trypsin (Sigma). The virus supernatants were collected as virus stocks used in the experiments. Virus titers were determined using the TCID50 as described previously.

### Example 7

### Mouse Immunization

Vaccine potency assays were carried out by mouse immunization. Balb/c mice were obtained from BioLASCO (Taipei, Taiwan). 25 μg of ΔSP-GG-VP1-C VLP and an equal mole amount of VP1 protein were diluted with KCl buffer and mixed with or without 5 ng LPS as adjuvant. Eight-week-old female mice were immunized with VLP (n=4), VP1 (n=3), VLP+adjuvant (n=4), and VP1+adjuvant (n=3) by footpad injection and boosted at day 7. Sera samples were collected by retro-orbital sampling at day 0, 7, and 14 for monitoring the immune response.

### Example 8

### Serological Assay

Antigen-specific antibodies from the immunized mice were examined by Western blot and ELISA. 5 μg of ΔSP-GG-VP1-C VLP and VP1 were fractionated by 10% SDS-PAGE before being transferred to a PVDF membrane (Bio-Rad), and subsequently probed with antisera (1:500), followed by incubation with horseradish peroxidase (HRP)-conjugated goat anti-mouse IgG (H+L) (1:20,000; 50 μl/well, Jackson ImmunoResearch, Cat No. 115-035-044). Membranes were developed with Western Chemiluminescent HRP Substrate (ECL) (Millipore) and exposed to X-ray film (FUJIFILM).

The titers of total anti-EV71, VLP1, and VLP2 IgG in sera were measured by ELISA. Each well of the plates was coated with 10 ng VLP antigens (diluted in 0.1 M NaHCO₃) and incubated at 4° C. overnight. After washes with PBST (0.1% Tween 20 in PBS) buffer, the wells were blocked with 200 μl of PBST containing 1% BSA at RT for 60 minutes. After washes, antisera were two-fold serially diluted (100-3,200 dilutions) and added into wells (50 μl/well). Plates were incubated at room temperature (RT) for 60 min and washed prior to addition of HRP-conjugated goat anti-mouse IgG (1:20,000; 50 μl/well). One hundred microliter of TMB (Millipore) was added for color development for 5-15 min. 50 μL H₂SO₄ (2 N) was added to stop the reaction and OD_{450/750} was measured by microplate reader (TECAN).

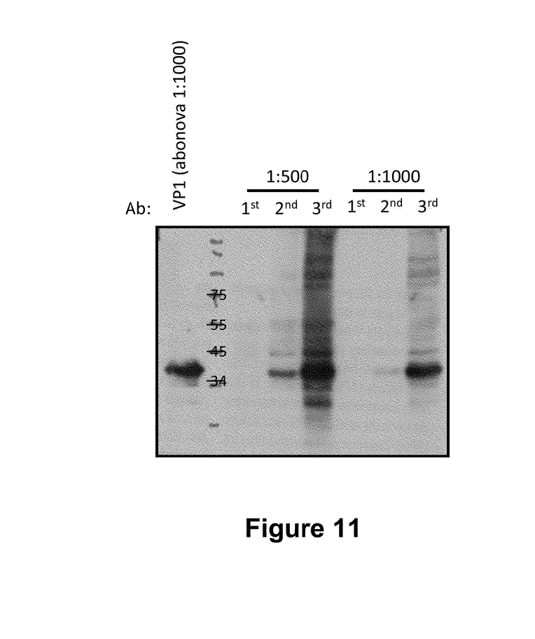

FIG. 11 illustrates a Western blot analysis of antisera obtained from mice immunized at different injection times with S-VP1-S VLPs.

In addition, female Balb/c mice were immunized with 25 μg of ΔSP-GG-VP1-C VLP and an equal mole amount of VP1 protein with or without 5 ng LPS as adjuvant. Sera was collected at day 0 (pre-immune) and day 14 post-immunization. The titer of anti-VLP IgG was determined by ELISA. The results are shown in the following table:

LPS (5 ng)

| Antigen | Pre-immune | 2^{nd} | Pre-immune | 2^{nd} |
|---|---|---|---|---|
| VP1 (n = 3) | <100 | <100 | <100 | <100 |
| VLP (n = 4) | <100 | 200 | <100 | 800 |

### Example 9

### Microneutralization Assay for EV71

Microneutralization assays were performed against EV71 in RD cells. Briefly, sera were heat-inactivated at 56° C. for 30 min and serially two-fold diluted from 1:10 to 1:1280 and mixed with an equal volume of EV71 virus (100 TCID₅₀/50 μl) in 96-well plates. After incubation at 37° C. for 1 h for virus neutralization, the serum-virus mixture was added into RD cells and incubated for 3-4 days for cellular cytopathic effects (CPE) observation or 39 h for ELISA test to detect virus antigen. For the neutralization-ELISA (Nt-ELISA) test, RD cells were fixed with 80% cold acetone and air dried. The air-dried plates were rehydrated with PBST to detect EV71. Rabbit polyclonal antibody against EV71 VP1 was used as the primary antibody (1:4000) and peroxidase-conjugated goat anti-rabbit IgG (1:20,000) (Jackson Immunoreserch) was used as the secondary antibody diluted in PBST containing 3% BSA. The optical densities (ODs) were read at 450 nm using TMB for color development.

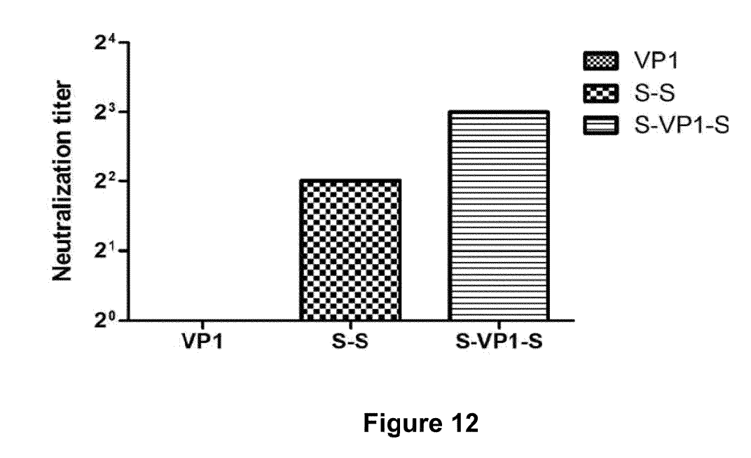

FIG. 12 illustrates the results of a microneutralization assay for EV71 using S-VP1-S VLPs.

### Example 10

### Microneutralization Assay for H1N1

Microneutralization assays will be performed against H1N1 in MDCK cells. Briefly, sera will be heat-inactivated at 56° C. for 30 min and serially two-fold diluted from 1:10 to 1:1280 and mixed with equal volume of H1N1 virus (100 TCID₅₀/50 μl) in 96-well plates. After incubation at 37° C. for 1 h for virus neutralization, the serum-virus mixture will be added into MDCK cells and incubated for 39 h. After incubation, MDCK cells will be fixed with 80% cold acetone and air dried. The air-dried plates will be rehydrated with PBST, to detect H1N1. Biotinylated monoclonal antibody against H1N1 nuclear protein (NP) will be used as the primary antibody (1:2000) (Millipore) and peroxidase-conjugated streptavidin (1:75,000) will be used as the secondary antibody diluted in PBST containing 1% BSA. The optical densities (ODs) will be read at 450 nm using TMB for color development.

### Example 11

### Additional Constructs

The following recombinant proteins of Examples 11-1 through 11-29 were generated using the VLP template as described in Examples 1-3:

| Example | Name | VP1 | L1 | Ag | L2 | VP2 |
|---|---|---|---|---|---|---|
| 11-1 | HBcS-GG-VP1 | HBc | G | VP1 | G | S |
| 11-2 | SHBs-GG-VP1 | S | G | VP1 | G | HBs |
| 11-3 | SHBs-EG-VP1 | S | E | VP1 | G | HBs |
| 11-4 | SHBs-RG-VP1 | S | R | VP1 | G | HBs |
| 11-5 | HBcHBs-GG-HA1 | HBc | G | HA1 | G | HBs |
| 11-6 | HBcHBs-GE-HA1 | HBc | G | HA1 | E | HBs |
| 11-7 | HBcHBs-EG-HA1 | HBc | E | HA1 | G | HBs |
| 11-8 | HBcHBs-GG-VP1 | HBc | G | VP1 | G | HBs |
| 11-9 | HBsHBc-GG-HA1 | HBs | G | HA1 | G | HBc |
| 11-10 | HBsHBc-GE-HA1 | HBs | G | HA1 | E | HBc |
| 11-11 | HBsHBc-EG-HA1 | HBs | E | HA1 | G | HBc |
| 11-12 | HBsHBc-EE-HA1 | HBs | E | HA1 | E | HBc |
| 11-13 | HBsP-GG-VP1 | HBs | G | VP1 | G | P |
| 11-14 | HBsP-GG-VP1 | HBs | G | VP1 | E | P |
| 11-15 | HBsP-GR-VP1 | HBs | G | VP1 | R | P |
| 11-16 | HBsP-EG-VP1 | HBs | E | VP1 | G | P |
| 11-17 | HBsP-ER-VP1 | HBs | E | VP1 | R | P |
| 11-18 | HBsP-RG-VP1 | HBs | R | VP1 | G | P |
| 11-19 | HBsP-RE-VP1 | HBs | R | VP1 | E | P |
| 11-20 | HBsP-RR-VP1 | HBs | R | VP1 | R | P |
| 11-21 | HBsP-EG-HA1 | HBs | E | HA1 | G | P |
| 11-22 | HBsP-EE-HA1 | HBs | E | HA1 | E | P |
| 11-23 | HBsP-ER-HA1 | HBs | E | HA1 | R | P |
| 11-24 | HBsP-GR-HA1 | HBs | G | HA1 | R | P |
| 11-25 | HBsP-RG-HA1 | HBs | R | HA1 | G | P |
| 11-26 | HBsP-RE-HA1 | HBs | R | HA1 | E | P |
| 11-27 | HBsHBs-EG-HA1 | HBs | E | HA1 | G | HBs |
| 11-28 | HBsHBs-RG-HA1 | HBs | R | HA1 | G | HBs |
| 11-29 | HBsHBs-GG-HA1 | HBs | G | HA1 | G | HBs |

### Example 12

### VLP Purification and Characterization

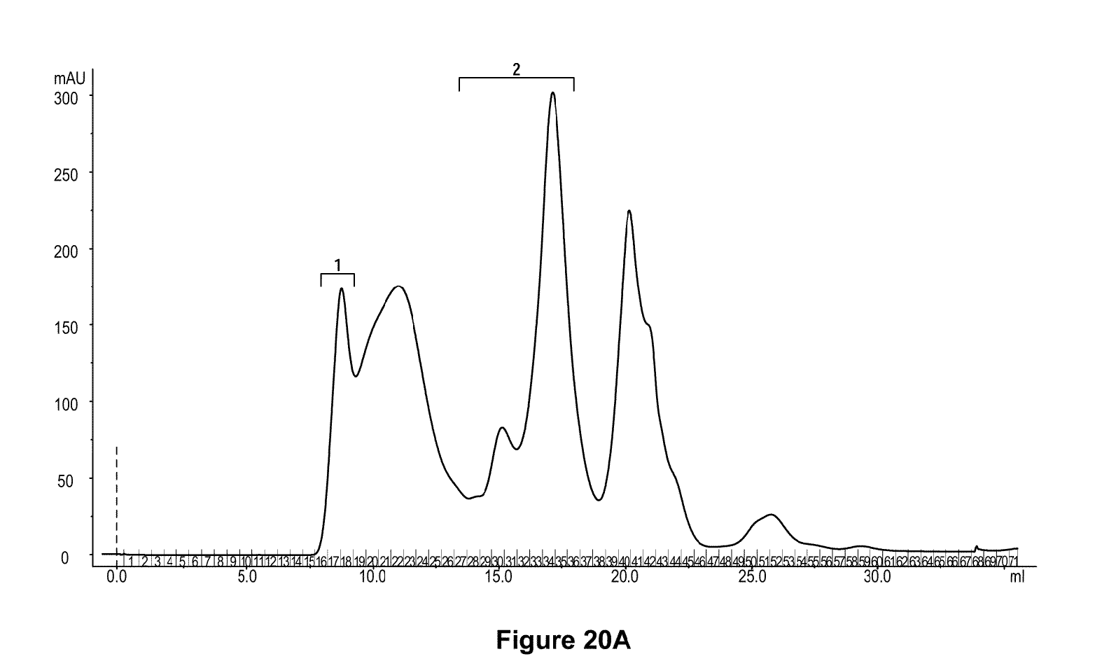

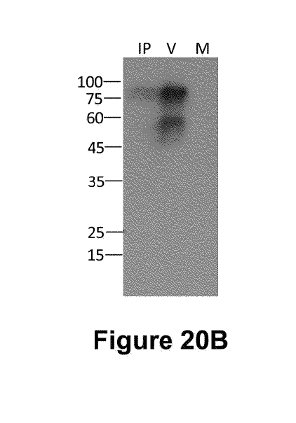

VLP formation of the recombinant proteins described in Example 11 was characterized by sucrose density gradient and size-exclusion chromatography. The VLPs of Example 11-1 (HBcS-GG-VP1) were purified as described in Example 4, except that VLPs in the crude extract were purified by affi-Streptactin chromatograpy (Strep-Tactin Superflow Plus, Qiagen Gmbh). Size-exclusion chromatography data for HBcHBs-GG-HA1 VLPs (Example 11-5) are depicted in FIG. 20. For all other VLPs from Example 11 that were formed with non-envelope structural proteins, the VLP purification and characterization method described in Example 4 was used. For VLPs from Example 11 formed with envelope structural proteins, the following methods were used.

Cell Lysis and Purification of HBsHBs-GG-HA1 (Example 11-29) Protein.

Yeast cells were harvested by centrifugation at 3,000×g for 5 minutes and resuspended in lysis buffer (20 mM Phosphate buffer pH 7.2, 5 mM EDTA, 150 mM NaCl, 1 mM PMSF, and 8% glycerol) with four-fold volume of wet cells at 4° C. Cells were broken by French press with pressure of 20,000 psi. The lysate was spun at 4° C. and 15,000×g for 15 minutes, and the membranes were washed twice with the lysis buffer (without EDTA). HBsHBs-GG-HA1 was extracted from the membranes at 30° C. for 16 hours in a volume of membrane extraction buffer (20 mM Phosphate buffer pH 7.2, 500 mM NaCl, 2% Tween-20) equal to the volume of lysis buffer. The membrane extract was separated from the cell debris by centrifugation. The supernatant after the centrifugation was adjusted to 1% Tween-20 using membrane extraction buffer without Tween-20. The extraction was applied to cobalt-based immobilized metal affinity chromatography (IMAC) (Clontech) for 4 hours at 4° C. according to the manufacturer's protocol, with the elution carried out with 250 mM imidazole. Fractions were subjected to 10% SD-PAGE and Western blot analysis.

Potassium Thiocyanate (KSCN) Treatment and Maturation of HBsHBs-GG-HA1 (Example 11-29) Protein.

The HBsHBs-GG-HA1 positive fractions were pooled, concentrated, and dialyzed against PBS (pH 7.2). The target protein was treated with 3 M KSCN at 4° C. for 16 hours, followed by buffer exchange into PBS. The KSCN-treated protein was maturated at 37° C. for 3 days and used for immunization studies.

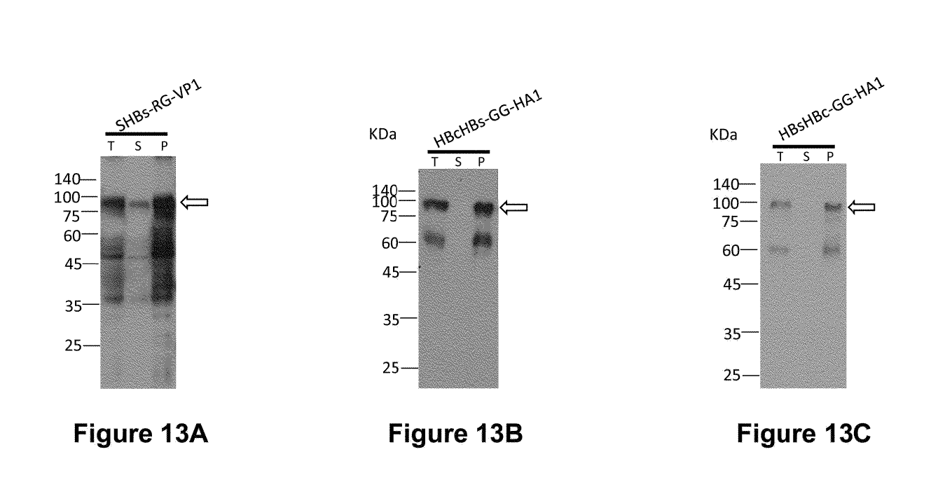

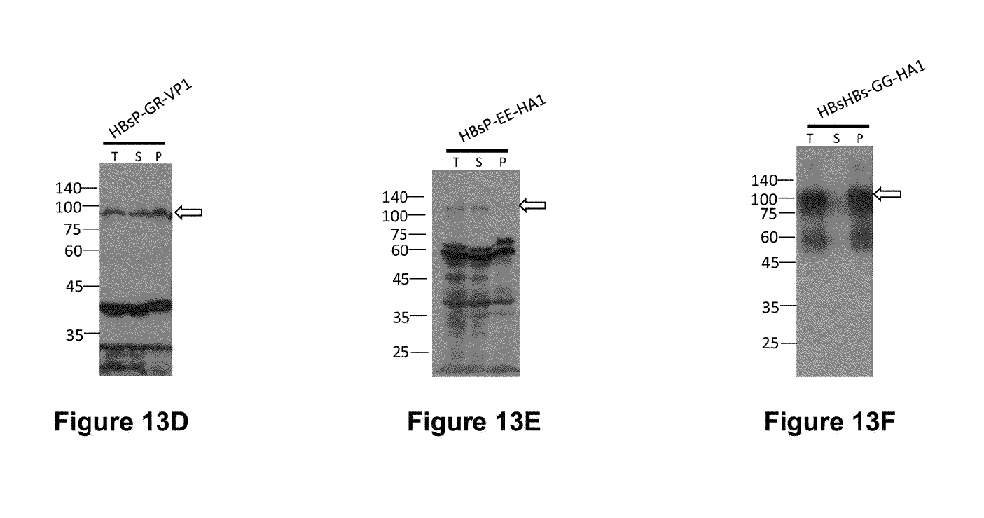

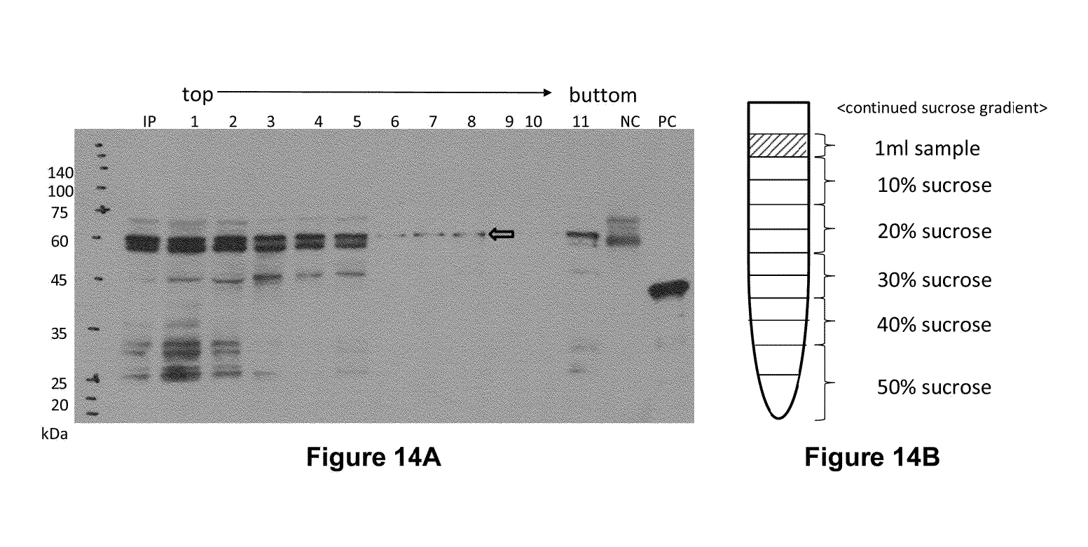

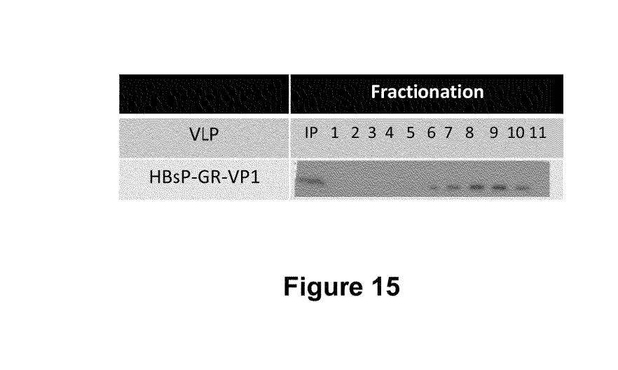

Expression of Examples 11-4, 11-5, 11-9, 11-15, 11-22, and 11-29 is shown in FIGS. 13A, 13B, 13C, 13D, 13E, and 13F, respectively. The results of sucrose gradient analysis of a HBcHBs-GG-VP1 (Example 11-8) VLP construct and the VLP construct of Example 11-15 are depicted in FIGS. 14A-B and FIG. 15, respectively.

### Example 13

### Transmission Electron Microscopy (TEM)

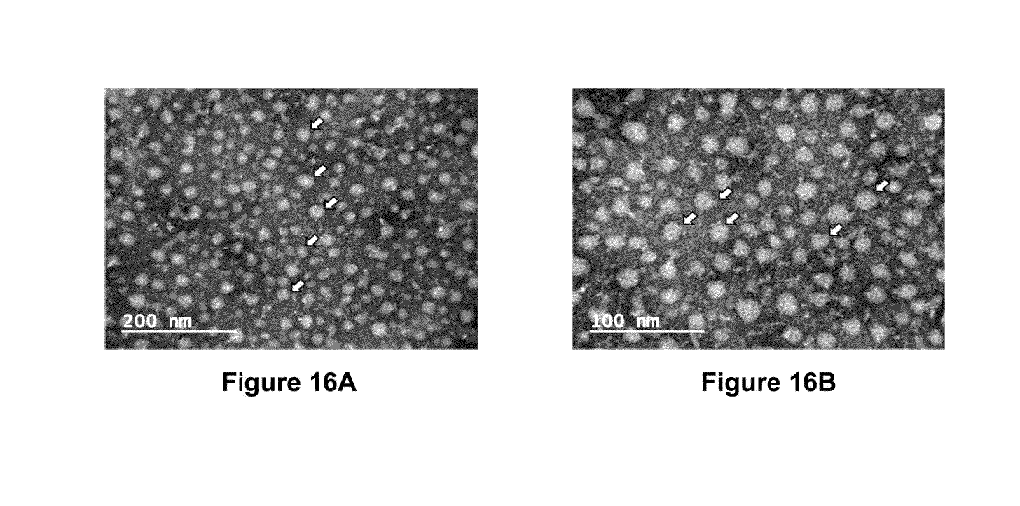

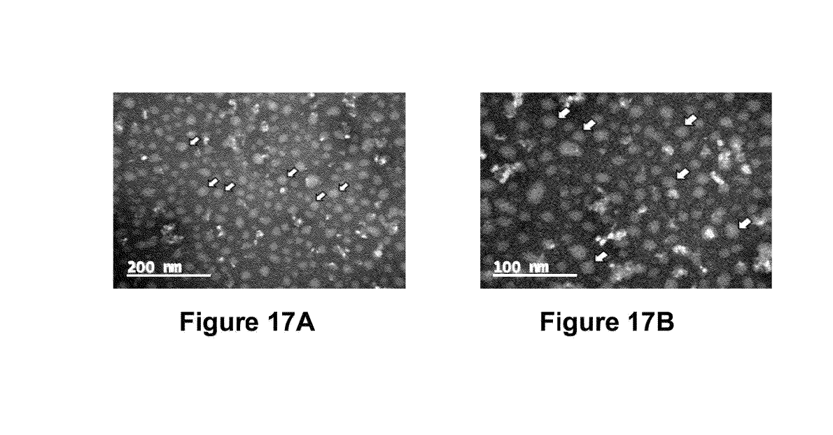

The particle size and morphology of the VLPs produced in Example 12 were characterized by TEM. Purified VLPs (0.04 mg/mL) were adsorbed onto formvar/carbon-coated copper grids (Electron Microscope Science) then negatively stained with 1% phosphotungstic acid. These samples were imaged using a JEOL JEM-1200EX II Transmission Electron Microscope. Transmission electron micrographs illustrating the morphology of structures of the VLP constructs of Examples 11-1 and 11-29 are provided in FIGS. 16A-B and FIGS. 17A-B, respectively.

### Example 14

### Mouse Immunization

Vaccine potency assays were carried out for the VLPs produced in Example 12 by mouse immuniziation. Balb/c mice were obtained form BioLASCO (Taipei, Taiwan).

HBsHBs-GG-HA1 (Example 11-29).

10 μg of HBsHBs-GG-HA1 VLPs and an equal mole amount of HA1 protein (4.8 μg) were diluted with PBS buffer and mixed with Alhydrogel adjuvant 2% (alum, InvivoGen) at a volume ratio of 1:1 for 5 minutes to allow the adjuvant to adsorb the antigen. 8-week old female mice were immunized with VLP (n=4), VLP+alum (n=4), HA1 (n=4), and HA1+alum (n=4) by intraperitoneal injection and boosted 3 doses at 7-day intervals. Sera were collected by retro-orbital sampling at day 0, 7, 14, 21, and 28, and stored at −20° C. before being used.

### Example 15

### Serological Assay

The titers of antigen specific for total IgG in antisera from Example 14 were examined by ELISA. Each well of plates was coated with antigen (diluted in 0.1 M NaHCO₃) and incubated at 4° C. overnight. After washes with PBST buffer (0.1% Tween 20 in PBS), the wells were blocked with 200 μL of PBST containing 1% BSA at room temperature for 60 minutes. After washes, antisera were two-fold serially diluted and added into wells (50 μL/well). Plates were incubated at room temperature for 60 minutes and washed prior to addition of HRP-conjugated goat anti-mouse IgG (1:20,000, 50 μL/well). 100 μL of TMB (Millipore) was added for color development for 5-15 minutes. 50 μL H₂SO₄ (2 N) was added to stop the reaction and OD_{450/750} was measured by microplate reader (TECAN).

Results for the immunogenicity of the VLP construct of Example 11-29 (HBsHBs-GG-HA1) are shown in the following table. The titer of anti-HBsHBs-GG-HA1 VLP IgG and anti-flu H1N1 virus were determined by ELISA.

| | Total anti-VLP IgG | | anti-flu H1N1 IgG | |
|---|---|---|---|---|
| | Pre-immune | | Pre-immune | |
| Immunization | serum | antiserum | serum | antiserum |
| VLP (n = 4) | <100 | 1600 | <100 | 1600 |
| HA1 (n = 4) | <100 | <100 | <100 | <100 |
| VLP + Alum | <100 | 12800 | <100 | 25600 |
| (n = 4) | | | | |
| HA1 + Alum | <100 | 12800 | <100 | 25600 |
| (n = 4) | | | | |

### Example 16

### Microneutralization Assay for H1N1

Cell Lines and Virus Strains.

MDCK cells were passaged in DMEM supplemented with 10% FBS, 1% L-glutamine, 1% penicillin/streptomycin, and 1% sodium pyruvate (Caisson) in a humidified atmosphere at 37° C. and 5% CO₂. The H1N1 virus (A/Taiwan/80813/2013), which was obtained form the Taiwan CDC, was propagated in MDCK cells with serum free DMEM supplemented with 2 μg/mL TPCK-trypsin (Sigma). The virus supernatants were collected as virus stocks used in the experiments. Virus titers were determined by plaque assay.

Assay.

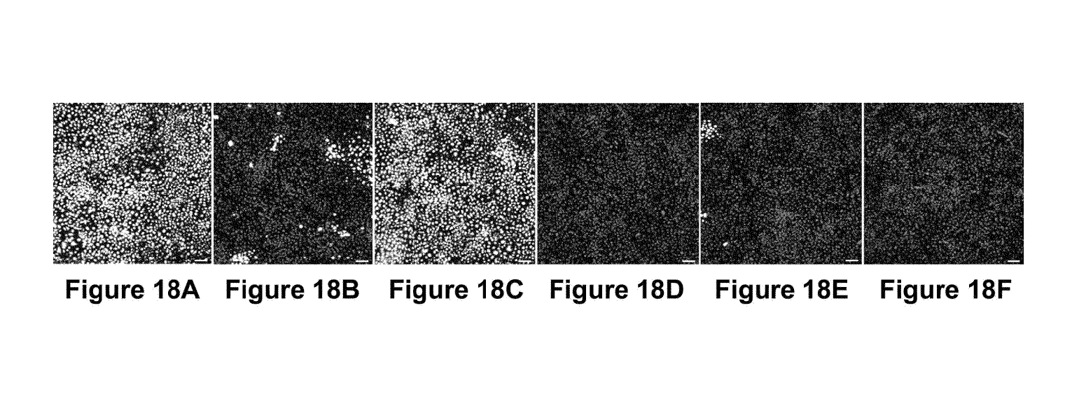

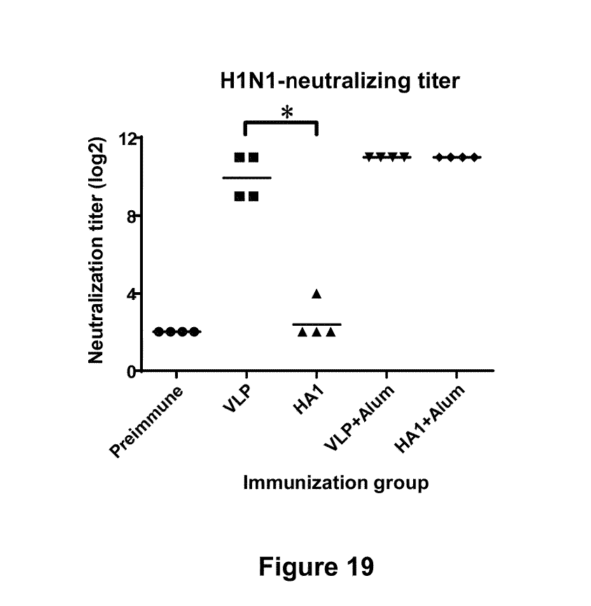

Sera from Example 14 was heat-inactivated at 56° C. for 30 minutes and serially two-fold diluted from 1:8 to 1:13,1072 (2¹⁷) and mixed with an equal volume of H1N1 virus (33 pfu/10 μL) in 384-well plates. After incubation at 37° C. for 1 hour for virus neturalization, the serum-virus mixture was added to MDCK cells. After virus adsorption for 1 hour, the mixture was discarded and the cells were washed with PBS. The diluted influenza virus was then added at 30 μL/well. The cells were then fixed with 4% paraformaldehyde for 30 minutes at 16 hpi, followed by 0.2% triton-X100 permeabilization for 10 minutes. The plates were washed with PBS and blocked with 0.5% BSA/PBS for 1 hour. To detect H1N1, a FITC-conjugated monoclonal antibody against H1N1 nuclear protein (NP) (Millipore) and Hoeschst (Sigma) were diluted in 0.5% BSA/PBS at 0.66 and 2 μL/mL separately. The fluorescence images were scanned and quantified by ImageXpress® Micro XL High-Content Image System (Molecular Devices). The highest dilution that produces 50% neutralization was recorded as the serum H1N1-neutralizing titer. Results are presented in FIGS. 18A-F and FIG. 19.

### Example 17

### Additional Constructs to be Generated

The following recombinant proteins of Examples 17-1 through 17-48 will be generated using the VLP template and tested as described above. For example, VLP named SHBs-GG-VP1 will be created by cloning the S domain of the Norovirus VP1 protein into the template using XhoI+NheI, cloning the N-terminal flexible linker (GGGGS)₃ (SEQ ID NO: 29) into the template using NheI+NdeI, cloning the VP1 antigen into the template using NdeI+PstI, cloning the C-terminal flexible linker (GGGGS)₃ (SEQ ID NO: 29) into the template using PstI+KpnI, and cloning the small BHV-derived surface antigen into the template using KpnI+SpeI.

| Example | Name | VP1 | L1 | Ag | L2 | VP2 |
|---|---|---|---|---|---|---|
| 17-1 | SHBs-GE-VP1 | S | G | VP1 | E | HBs |
| 17-2 | SHBs-GR-VP1 | S | G | VP1 | R | HBs |
| 17-3 | SHBs-EE-VP1 | S | E | VP1 | E | HBs |
| 17-4 | SHBs-ER-VP1 | S | E | VP1 | R | HBs |
| 17-5 | SHBs-RE-VP1 | S | R | VP1 | E | HBs |
| 17-6 | SHBs-RR-VP1 | S | R | VP1 | R | HBs |
| 17-7 | SHBs-GG-HA1 | S | G | HA1 | G | HBs |
| 17-8 | SHBs-GE-HA1 | S | G | HA1 | E | HBs |
| 17-9 | SHBs-GR-HA1 | S | G | HA1 | R | HBs |
| 17-10 | SHBs-EG-HA1 | S | E | HA1 | G | HBs |
| 17-11 | SHBs-EE-HA1 | S | E | HA1 | E | HBs |
| 17-12 | SHBs-ER-HA1 | S | E | HA1 | R | HBs |
| 17-13 | SHBs-RG-HA1 | S | R | HA1 | G | HBs |
| 17-14 | SHBs-RE-HA1 | S | R | HA1 | E | HBs |
| 17-15 | SHBs-RR-HA1 | S | R | HA1 | R | HBs |
| 17-16 | S-HBs-GG-M2 | S | G | M2 | G | HBs |
| 17-17 | S-HBs-GE-M2 | S | G | M2 | E | HBs |
| 17-18 | S-HBs-GR-M2 | S | G | M2 | R | HBs |
| 17-19 | S-HBs-EG-M2 | S | E | M2 | G | HBs |
| 17-20 | S-HBs-EE-M2 | S | E | M2 | E | HBs |
| 17-21 | S-HBs-ER-M2 | S | E | M2 | R | HBs |
| 17-22 | S-HBs-RG-M2 | S | R | M2 | G | HBs |
| 17-23 | S-HBs-RE-M2 | S | R | M2 | E | HBs |
| 17-24 | S-HBs-RR-M2 | S | R | M2 | R | HBs |
| 17-25 | HBsHBs-GG-VP1 | HBs | G | VP1 | G | HBs |
| 17-26 | HBsHBs-GE-VP1 | HBs | G | VP1 | E | HBs |
| 17-27 | HBsHBs-GR-VP1 | HBs | G | VP1 | R | HBs |
| 17-28 | HBsHBs-EG-VP1 | HBs | E | VP1 | G | HBs |
| 17-29 | HBsHBs-EE-VP1 | HBs | E | VP1 | E | HBs |
| 17-30 | HBsHBs-ER-VP1 | HBs | E | VP1 | R | HBs |
| 17-31 | HBsHBs-RG-VP1 | HBs | R | VP1 | G | HBs |
| 17-32 | HBsHBs-RE-VP1 | HBs | R | VP1 | E | HBs |
| 17-33 | HBsHBs-RR-VP1 | HBs | R | VP1 | R | HBs |
| 17-34 | HBsHBs-GE-HA1 | HBs | G | HA1 | E | HBs |
| 17-35 | HBsHBs-GR-HA1 | HBs | G | HA1 | R | HBs |
| 17-36 | HBsHBs-EE-HA1 | HBs | E | HA1 | E | HBs |
| 17-37 | HBsHBs-ER-HA1 | HBs | E | HA1 | R | HBs |
| 17-38 | HBsHBs-RE-HA1 | HBs | R | HA1 | E | HBs |
| 17-39 | HBsHBs-RR-HA1 | HBs | R | HA1 | R | HBs |
| 17-40 | HBsHBs-GG-M2 | HBs | G | M2 | G | HBs |
| 17-41 | HBsHBs-GE-M2 | HBs | G | M2 | E | HBs |
| 17-42 | HBsHBs-GR-M2 | HBs | G | M2 | R | HBs |
| 17-43 | HBsHBs-EG-M2 | HBs | E | M2 | G | HBs |
| 17-44 | HBsHBs-EE-M2 | HBs | E | M2 | E | HBs |
| 17-45 | HBsHBs-ER-M2 | HBs | E | M2 | R | HBs |
| 17-46 | HBsHBs-RG-M2 | HBs | R | M2 | G | HBs |
| 17-47 | HBsHBs-RE-M2 | HBs | R | M2 | E | HBs |
| 17-48 | HBsHBs-RR-M2 | HBs | R | M2 | R | HBs |

Although the present invention has been described in the context of particular examples and embodiments, those skilled in the art will recognize equivalent embodiments that are also included within the scope of the claims in the following listing.

What is claimed is:

1. A fusion protein comprising:
(a) V1-L1-Ag-L2-V2, wherein
V1 is an N-terminal viral structural protein,
L1 is an N-terminal linker,
Ag is an antigen or antigenic fragment of a pathogen,
L2 is a C-terminal linker, and
V2 is a C-terminal viral structural protein, and
wherein each of V1 and V2 is, independently or together, capable of forming a virus-like particle;
(b) V1-L1-Ag-V2 or V1-Ag-L2-V2, wherein
V1 is an N-terminal viral structural protein,
L1 is an N-terminal linker,
Ag is an antigen or antigenic fragment of a pathogen,
L2 is a C-terminal linker, and
V2 is a C-terminal viral structural protein, and
wherein each of V1 and V2 is, independently or together, capable of forming a virus-like particle;
(c) V1-Ag-V2, wherein
V1 is an N-terminal viral structural protein,
Ag is an antigen or antigenic fragment of a pathogen,
V2 is a C-terminal viral structural protein, and
wherein each of V1 and V2 is, independently or together, capable of forming a virus-like particle;
(d) V1-L1-Ag-L2-V2, wherein
V1 is a fragment of an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and wherein V1 and V2 are the same;
(e) V1-L1-Ag-V2 or V1-Ag-L2-V2, wherein
V1 is a fragment of an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and wherein V1 and V2 are the same;
(f) V1-Ag-V2, wherein
V1 is a fragment of an N-terminal viral structural protein,
Ag is an antigen or antigenic fragment of a pathogen,
V2 is a fragment of a C-terminal viral structural protein, and
wherein each of V1 and V2 is, independently or together, capable of forming a virus-like particle, and wherein V1 and V2 are the same;
(g) V1-L1-Ag-L2-V2, wherein
V1 is a fragment of an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and
wherein V1 and V2 are from different proteins from the same virus;
(h) V1-L1-Ag-V2 or V1-Ag-L2-V2, wherein
V1 is a fragment of an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and
wherein V1 and V2 are from different proteins from the same virus;
(i) V1-Ag-V2, wherein
V1 is a fragment of an N-terminal viral structural protein,
Ag is an antigen or antigenic fragment of a pathogen,
V2 is a fragment of a C-terminal viral structural protein, and
wherein each of V1 and V2 is, independently or together, capable of forming a virus-like particle, and
wherein V1 and V2 are from different proteins from the same virus;
(j) V1-L1-Ag-L2-V2, wherein
V1 is a fragment of an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and
wherein V1 and V2 are from proteins of different viruses;
(k) V1-L1-Ag-V2 or V1-Ag-L2-V2, wherein
V1 is a fragment of an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and
wherein V1 and V2 are from proteins from different viruses;
(l) V1-Ag-V2, wherein
V1 is a fragment of an N-terminal viral structural protein,
Ag is an antigen or antigenic fragment of a pathogen,
V2 is a fragment of a C-terminal viral structural protein, and
wherein each of V1 and V2 is, independently or together, capable of forming a virus-like particle, and
wherein V1 and V2 are from proteins from different viruses;
(m) V1-L1-Ag-L2-V2, wherein
V1 is a fragment of an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and
wherein the fragments are from different portions of the same parent viral structural protein, and
wherein the combined amino acid sequence of V1 and V2 comprises less than the complete amino acid sequence of the parent viral structural protein;
(n) V1-L1-Ag-V2 or V1-Ag-L2-V2, wherein
V1 is a fragment of an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and
wherein the fragments are from different portions of the same parent viral structural protein, and
wherein the combined amino acid sequence of V1 and V2 comprises less than the complete amino acid sequence of the parent viral structural protein;
(o) V1-Ag-V2, wherein
V1 is a fragment of an N-terminal viral structural protein,
Ag is an antigen or antigenic fragment of a pathogen,
V2 is a fragment of a C-terminal viral structural protein, and
wherein each of V1 and V2 is, independently or together, capable of forming a virus-like particle, and
wherein the fragments are from different portions of the same parent viral structural protein, and
wherein the combined amino acid sequence of V1 and V2 comprises less than the complete amino acid sequence of the parent viral structural protein;
(p) V1-L1-Ag-L2-V2, wherein
V1 is an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and
wherein V2 is a fragment of V1;
(q) V1-L1-Ag-L2-V2, wherein
V1 is a fragment of an N-terminal viral structural protein,
L1 is an N-terminal linker,
Ag is an antigen or antigenic fragment of a pathogen,
L2 is a C-terminal linker, and
V2 is a C-terminal viral structural protein, and
wherein each of V1 and V2 is, independently or together, capable of forming a virus-like particle, and
wherein V1 is a fragment of V2;
(r) V1-L1-Ag-V2 or V1-Ag-L2-V2, wherein
V1 is an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and
wherein V2 is a fragment of V1;
(s) V1-L1-Ag-V2 or V1-Ag-L2-V2, wherein
V1 is a fragment of an N-terminal viral structural protein
L1 is an N-terminal linker,
Ag is an antigen or antigenic fragment of a pathogen,
L2 is a C-terminal linker, and
V2 is a C-terminal viral structural protein, and
wherein each of V1 and V2 is, independently or together, capable of forming a virus-like particle, and
wherein V1 is a fragment of V2;
(t) V1-Ag-V2, wherein
V1 is an N-terminal viral structural protein,
Ag is an antigen or antigenic fragment of a pathogen,
V2 is a fragment of a C-terminal viral structural protein, and
wherein each of V1 and V2 is, independently or together, capable of forming a virus-like particle, and
wherein V2 is a fragment of V1;
(u) V1-Ag-V2, wherein
V1 is a fragment of an N-terminal viral structural protein,
Ag is an antigen or antigenic fragment of a pathogen,
V2 is a C-terminal viral structural protein, and
wherein each of V1 and V2 is, independently or together, capable of forming a virus-like particle, and
wherein V1 is a fragment of V2;
(v) V1-L1-Ag-L2-V2, wherein
V1 is an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and
wherein V2 is a fragment of a different protein from the same virus as V1;
(w) V1-L1-Ag-L2-V2, wherein
V1 is a fragment of an N-terminal viral structural protein,
L1 is an N-terminal linker,
Ag is an antigen or antigenic fragment of a pathogen,
L2 is a C-terminal linker, and
V2 is a C-terminal viral structural protein, and
wherein each of V1 and V2 is, independently or together, capable of forming a virus-like particle, and
wherein V1 is a fragment of a different protein from the same virus as V2;
(x) V1-L1-Ag-V2 or V1-Ag-L2-V2, wherein
V1 is an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and
wherein V2 is a fragment of a different protein from the same virus as V1;
(y) V1-L1-Ag-V2 or V1-Ag-L2-V2, wherein
V1 is a fragment of an N-terminal viral structural protein,
L1 is an N-terminal linker,
Ag is an antigen or antigenic fragment of a pathogen,
L2 is a C-terminal linker, and
V2 is a C-terminal viral structural protein, and
wherein each of V1 and V2 is, independently or together, capable of forming a virus-like particle, and
wherein V1 and V2 are from different proteins from the same virus;
(z) V1-Ag-V2, wherein
V1 is an N-terminal viral structural protein,
Ag is an antigen or antigenic fragment of a pathogen,
V2 is a fragment of a C-terminal viral structural protein, and
wherein each of V1 and V2 is, independently or together, capable of forming a virus-like particle, and
wherein V2 is a fragment of a different protein from the same virus as V1;
(aa) V1-Ag-V2, wherein
V1 is a fragment of an N-terminal viral structural protein,
Ag is an antigen or antigenic fragment of a pathogen,
V2 is a C-terminal viral structural protein, and
wherein each of V1 and V2 is, independently or together, capable of forming a virus-like particle, and
wherein V1 is a fragment of a different protein from the same virus as V2;
(bb) V1-L1-Ag-L2-V2, wherein
V1 is an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and
wherein V2 is a fragment of a protein from a different virus than V1;
(cc) V1-L1-Ag-L2-V2, wherein
V1 is a fragment of an N-terminal viral structural protein,
L1 is an N-terminal linker,
Ag is an antigen or antigenic fragment of a pathogen,
L2 is a C-terminal linker, and
V2 is a C-terminal viral structural protein, and
wherein each of V1 and V2 is, independently or together, capable of forming a virus-like particle, and
wherein V1 is a fragment of a protein from a different virus than V2;
(dd) V1-L1-Ag-V2 or V1-Ag-L2-V2, wherein
V1 is an N-terminal viral structural protein,
L1 is an N-terminal linker,
Ag is an antigen or antigenic fragment of a pathogen,
L2 is a C-terminal linker, and
V2 is a fragment of a C-terminal viral structural protein, and
wherein each of V1 and V2 is, independently or together, capable of forming a virus-like particle, and
wherein V2 is a fragment of a protein from a different virus than V1;
(ee) V1-L1-Ag-V2 or V1-Ag-L2-V2, wherein
V1 is a fragment of an N-terminal viral structural protein,
L1 is an N-terminal linker,
Ag is an antigen or antigenic fragment of a pathogen,
L2 is a C-terminal linker, and
V2 is a C-terminal viral structural protein, and
wherein each of V1 and V2 is, independently or together, capable of forming a virus-like particle, and
wherein V1 is a fragment of a protein from a different virus than V2;
(ff) V1-Ag-V2, wherein
V1 is an N-terminal viral structural protein,
Ag is an antigen or antigenic fragment of a pathogen,
V2 is a fragment of a C-terminal viral structural protein, and
wherein each of V1 and V2 is, independently or together, capable of forming a virus-like particle, and
wherein V2 is a fragment of a protein from a different virus than V1; or
(gg) V1-Ag-V2, wherein
V1 is a fragment of an N-terminal viral structural protein,
Ag is an antigen or antigenic fragment of a pathogen,
V2 is a C-terminal viral structural protein, and
wherein each of V1 and V2 is, independently or together, capable of forming a virus-like particle, and
wherein V1 is a fragment of a protein from a different virus than V2.

2. The fusion protein of claim 1, wherein Ag is selected from the group consisting of:
(a) an antigenic peptide, polypeptide, or protein from a viral pathogen,
(b) an antigenic peptide, polypeptide, or protein from a bacterial pathogen,
(c) an antigenic peptide, polypeptide, or protein from a parasitic pathogen,
(d) an antigenic peptide, polypeptide, or protein from a fungal pathogen, and
(e) an antigenic peptide, polypeptide, or protein from a prion.

3. The fusion protein of claim 1, wherein V1 and V2 are selected from the group consisting of: a viral capsid protein and a viral envelope protein.

4. The fusion protein of claim 1, wherein V1 and V2 are selected from the group consisting of:
(a) HBc of HBV virus,
(b) the small HBV-derived surface antigen (HBsAg),
(c) the S domain of Norovirus capsid protein VP1,
(d) the P domain of Norovirus capsid protein VP1,
(e) Human Rotavirus VP2,
(f) Human Rotavirus VP6,
(g) the L1 major capsid protein of human papillomavirus,
(h) the VP1 of human polyomavirus,
(i) the VP1 of human JC virus,
(j) the VP2 of human adeno-associated virus 2,
(k) the VP3 of human adeno-associated virus 2,
(l) the S and P1 domain of Hepatitis E virus capsid protein VP1, and
(m) the P2 domain of Hepatitis E virus capsid protein VP1.

5. The fusion protein of claim 1, wherein V1 and V2 of (a), (b), or (c) are the same viral structural protein.

6. The fusion protein of claim 1, wherein V1 and V2 of (a), (b), or (c) are different viral structural proteins of the same virus.

7. The fusion protein of claim 1, wherein V1 and V2 of (a), (b), or (c) are viral structural proteins of different viruses.

8. The fusion protein of claim 1, wherein at least one of V1 and V2 is immunogenic in the fusion protein, in the virus-like particle, or in both the fusion protein and the virus-like particle.

9. The fusion protein of claim 1, wherein both V1 and V2 are immunogenic in the fusion protein, in the virus-like particle, or in both the fusion protein and the virus-like particle.

10. The fusion protein of claim 1, wherein at least one of L1 and L2 of (a), (b), (d), (e), (g), (h), (j), (k), (m), (n), (p)-(s), (v)-(y), or (bb)-(ee) is selected from the group consisting of: a flexible linker, a cleavable linker, a rigid linker, and an unstructured random coil peptide.

11. The fusion protein of claim 1, wherein L1 and L2 of (a), (d), (g), (j), (m), (p), (q), (v), (w), (bb), or (cc) are the same linker.

12. The fusion protein of claim 1, wherein L1 and L2 of (a), (d), (g), (j), (m), (p), (q), (v), (w), (bb), or (cc) are different linkers.

13. A recombinant nucleic acid expression vector comprising a polynucleotide encoding a fusion protein of claim 1.

14. A host cell comprising the recombinant nucleic acid expression vector of claim 13.

15. A virus-like particle comprising the fusion protein of claim 1.

16. A pharmaceutical composition comprising the virus-like particle of claim 15 and a pharmaceutically acceptable carrier.

17. A pharmaceutical composition comprising the fusion protein of claim 1 and a pharmaceutically acceptable carrier.

18. A method of inducing an immune response in a mammalian subject comprising administering to the subject the pharmaceutical composition of claim 16 in an amount sufficient to generate an immune response in the subject.

19. A method of inducing an immune response in a mammalian subject comprising administering to the subject the pharmaceutical composition of claim 17 in an amount sufficient to generate an immune response in the subject.

20. A method for preparing virus-like particles, said method comprising culturing the host cell of claim 14 under conditions that permit expression of said fusion protein and assembly of said fusion protein to form said virus-like particles.
